(19) United States
(12) Patent Application Publication
(10) Pub. No.: US 2009/0030037 A1
(43) Pub. Date: Jan. 29, 2009

(54) Sustained Release Formulation and Dosing Schedules of Leukotriene Synthesis Inhibitor for Human Therapy

(75) Inventors: Mark E. Gurney, Grand Rapids, MI (US); Hakon Hakonarson, Malvern, PA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: deCODE Genetics ehf., Reykjavik (IS)

(21) Appl. No.: 11/912,248

(22) PCT Filed: Apr. 21, 2006
(86) PCT No.: PCT/US2006/015542

§ 371 Date: Sep. 22, 2008

Related U.S. Application Data
(60) Provisional application No. 60/673,981, filed on Apr. 21, 2005.

(51) Int. Cl. A61K 31/47; A61P 29/00

(52) U.S. Cl. 514/311

(57) ABSTRACT

The invention relates to materials and methods for therapy to inhibit the production of leukotrienes, and therapeutic and prophylactic applications thereof.

This application claims priority benefit of U.S. Provisional Patent Application No. 60/673,981 filed Apr. 21, 2005, which is incorporated by reference herein in its entirety.

### FIELD OF INVENTION

The invention relates to improved materials and methods for therapy to inhibit production of leukotrienes, and all therapeutic applications thereof.

### BACKGROUND

The end products of the leukotriene pathway are potent inflammatory lipid mediators derived from arachidonic acid. They can potentially contribute to development of atherosclerosis and destabilization of atherosclerotic plaques through lipid oxidation or other pro-inflammatory effects. Leukotriene C4 (LTC4), leukotriene D4 (LTD4), and leukotriene E4 (LTE4), are known to induce vasoconstriction. Allen et al., Circulation, 97:2406-2413 (1998) described a novel mechanism in which atherosclerosis is associated with the appearance of a leukotriene receptor(s) capable of inducing hyperactivity of human epicardial coronary arteries in response to LTC4 and LTD4. LTB4, on the other hand, is a strong pro-inflammatory agent. Variants of the 5-lipoxygenase (5-LO) Activating Protein (FLAP) gene and the leukotriene A4 hydrolase (LTA4H) gene have been associated with elevated risk for myocardial infarction in Icelandic, British and North American populations, as described in PCT Application Nos. PCT/US03/32805, PCT/US03/32556, PCT/US04/030582 and PCT/US05/03312.

Antagonists of leukotriene synthesis, such as antagonists of FLAP activity or production are being developed as therapeutics for inflammatory disease states, such as asthma, and cardiovascular disease states such as myocardial infarction and arthrosclerosis. However, a need exists for ways to maximize the efficacy of such therapy, and to minimize its side effects. A need also exists to maximize the convenience of such therapy, to improve compliance and reduce patient errors.

### SUMMARY OF INVENTION

The invention provides materials and methods to achieve and maintain steady state plasma concentration of a leukotriene synthesis inhibitor, such as DG-031, in a human, wherein these steady-state concentration ranges exhibit the most beneficial effects and reduce drug exposure, thereby achieving maximum efficiency and reducing the possibility of short term or long term drug side-effects. To achieve and maintain a steady-state plasma concentration of the leukotriene synthesis inhibitor, the invention provides for dosing schedules that are effective to attain this effect. Numerous compounds are described below in the compound section, and each represents an embodiment of the invention. For brevity, the invention is described in the context of one of the preferred embodiments, DG-031.

In the context of the invention, the term “dose” refers to a quantity of a therapeutic agent to be administered at one time, and the term. “dosing schedule” describes the time course and frequency during which multiple doses of a therapeutic agent are administered to a human or animal subject for therapeutic or prophylactic purposes. For example, doses of 500 mg of therapeutic agent might be administered on a two times per day dosing schedule, which would normally be administered approximately every twelve hours. Alternatively, doses of 250 mg of therapeutic agent might be administered on a three times per day dosing schedule, which would normally be administered approximately every eight hours. The invention provides dosing schedules which effectively achieve and maintain a steady state concentration of a leukotriene inhibitor such as DG-031, wherein the steady state concentration attains a desired therapeutic effect in a human and reduces the potential for adverse events. The invention also provides for methods of administering doses of DG-031 to a human according to a dosing schedule of the invention in order to attain the desired therapeutic effect in said human. Preferred embodiments involve doses and dosing schedules that are convenient to patients, e.g., with fewer daily doses.

A “steady state concentration” in a human subject receiving treatment is a concentration of therapeutic agent that is at a dynamic equilibrium, fluctuating periodically within a reasonably predictable and periodic range with the fluctuation determined by the dosing schedule. The invention provides for dosing schedules of DG-031 that attain a dynamic equilibrium of DG-031 within a desired range in the plasma of the individual receiving the doses according to the dosing schedule.

The term “peak concentration,” also referred to as “Cmax,” refers to the maximum concentration achieved in the steady state dynamic equilibrium, which can be visualized as the top of a peak or maxima on a graph of plasma concentration over time. With oral or other bolus dosing, the peak concentration usually occurs some time between doses, with the time depending on the route of administration and formulation. The “trough concentration,” also referred to as “Cmin,” refers to the minimum concentration achieved in the steady state dynamic equilibrium, which can be visualized as the minima on a graph of plasma concentration plotted over time. With oral or other bolus dosing, the trough concentration following a dose usually is observed at a time corresponding to immediately before administration of a new/next dose.

In the context of treatment of a single individual, a steady state concentration may be expected to fluctuate from day-to-day with variations in the individual's diet, level activity, state of health, co-administration of other medications, and the like. DG-031 is preferably administered with food to improve adsorption. For example, administration with a high fat meal caused a 150% increase in Cₘₐₓ, 30% increase in AUC, a shorter half-life and no effect on Tₘₐₓ. Alternatively, DG-031 is administered in the absence of food. The decision whether or not to administer with food may be based on the Cmax and Cmin observed following administration of a novel formulation where the Cmax and Cmin fall within the desired range. Many indications for the therapies described herein, such as prophylaxis for myocardial infarction, benefit from repeat dosing for weeks or months or years. In this context, the steady state concentration for an individual refers to an average concentration taken at multiple time points, to adjust for such fluctuation.

In the context of a treatment regimen, a steady state concentration refers to an average or mean (preferably, a geometric mean) dynamic equilibrium obtained from observations of a statistically representative number of individuals, taking into account factors such as sex, weight, race and age. Likewise, in the context of evaluating the properties of a particular formulation, such as a sustained or controlled release oral pill or capsule, a steady state concentration refers to an average or mean (preferably, a geometric mean) dynamic equilibrium obtained from observations of a statistically representative number of individuals, taking into account the same aforementioned factors.

One embodiment of the invention is a method of treating or preventing an inflammatory condition or disease in a human using a leukotriene inhibitor substance such as DG-031 or related compounds. For example, in one embodiment, the invention is a method of treating or preventing an inflammatory condition or disease in a human comprising administering doses of DG-031, or a pharmaceutically acceptable salt or ester or prodrug thereof, according to a dosing schedule that is effective to maintain a steady state DG-031 plasma concentration in a range of 6 μM to 31 μM (2.2 μg/ml to 11.2 μg/ml) in said human. Here and elsewhere, microgram measurements refer to the DG-031 compound. It will be understood that micromolar equivalents of DG-031 salts or esters will vary when converted to μg/ml or other mass units.

Another embodiment of the invention is a use of DG-031, or a pharmaceutically acceptable salt, ester, or pro-drug thereof, in the manufacture of a medicament for administration to a human for treatment or prophylaxis for an inflammatory disease or condition, so as to sustain a biological response in the treatment or prophylaxis of the disease or condition, wherein the medicament is formulated into a dose that is administered according to a dosing schedule that is effective to maintain a steady state DG-031 plasma concentration in a range of 6 μM to 31 μM (2.2 μg/ml to 11.2 μg/ml) in the human.

In a preferred embodiment, the doses of the methods and uses of the invention are formulated for oral administration and are administered orally. Preferably, the invention provides for dosing schedules that are no more than three times a day and no more than two times per day.

Measurements of plasma DG-031 can be carried out by methods that are standard in the art. In a preferred method of measuring plasma DG-031, plasma is separated from the blood samples from the treated patients and the protein in the plasma is precipitated by an organic solvent such as acetonitrile. The DG-031 is then measured by liquid chromatography and/or mass spectrometry and the measured concentration is compared to a standard curve.

The term “treating” refers to providing any measure of therapeutic benefit, such as reduction of symptoms, measurable improvement in therapeutically meaningful biological molecules, slowing of deterioration, or curing. The term “preventing” refers to any measure of preventative/prophylactic benefit. For example, effective prevention can be measured in an individual by a slowing or elimination of deterioration or a delay in an expected adverse event. Prevention is often more readily demonstrated in a clinical setting or population study that demonstrates that a population of individuals that receive a therapy suffer fewer adverse events, or survive longer, or suffer less severe adverse events, or enjoy any other benefit as a group that a physician would characterize as prophylactic or beneficial.

The term “prodrug” refers to a chemical entity that is metabolized in vivo into an active drug such as DG-031, such entities being designable and identifiable by pharmaceutical chemists.

A preferred human subject for treatment according to the invention is an adult human, particularly an adult humans suffering from an inflammatory disease and/or an adult human identified as being at risk for developing an inflammatory disease or condition. Treatment of adult humans identified as having or at risk for developing cardiovascular disease is specifically contemplated. An exemplary human subject of the invention is an individual who is at risk for suffering a myocardial infarction (MI) as indicated by elevated levels of a leukotriene or an inflammatory marker such as C-reactive protein (CRP) or myeloperoxidose (MPO). Another exemplary human subject of the invention is an individual who has suffered at least one myocardial infarction in the past. Another exemplary human subject is an individual identified as at-risk for MI due to a genetic predisposition, such as a predisposing single nucleotide polymorphism (SNP) or haplotype in a gene such as FLAP, LTA4H, or 5-LO. See PCT Application No. PCT/US03/32805, filed Oct. 16, 2003, PCT/US03/32556 filed Oct. 16, 2003, PCT/US04/030582 filed Sep. 17, 2004, PCT/US05/03312 filed Jan. 31, 2005 and PCT Application No. ______ filed Mar. 29, 2006 (attorney docket no. 30847/40807A), which are incorporated by reference herein in their entirety. Also see U.S. Publication No. US-2006-0019269-A1 filed Mar. 30, 2005 and U.S. patent application Ser. No. 11/270,804 (Publication No. ______) filed Nov. 9, 2006, which are incorporated by reference herein in their entirety.

An exemplary FLAP haplotypes that is associated with risk for MI is HapA, which is defined by allele G at marker SG13S25, allele T at marker SG13S114, allele G at marker SG13S89, and allele A at marker SG13S32 within the FLAP gene.

Another example of a FLAP haplotype that is associated with risk for MI is HapC, which is defined by the T allele of marker SG13S375, allele G of marker SG12S25, allele G of marker SG12S106 and allele A of marker SG12S32 within the FLAP gene. There are 4 additional variations of the HapC haplotype which comprise SNPs in addition to the T allele of SG13S375. HapC2 is defined by allele T of the SNPs SG13S375 and allele G of the SNP SG13S25. HapC3 is defined by allele T of the SNPs SG13S375 and allele G of the SNP SG13S25 and allele A of SNP SG13S32. HapC4-A is defined by allele G of the SNP SG13S106 in addition to allele T of the SNPs SG13S375, allele G of the SNP SG13S25 and allele A of SNP SG13S32. HapC4-B is defined by allele A of the SNP SG13S106 in addition to allele T of the SNPs SG13S375, allele G of the SNP SG13S25 and allele A of SNP SG13S32. HapC4-A correlates with HapA and HapB.

An exemplary LTA4H haplotype that is associated with risk for MI is HapK. HapK is defined by allele C of the SNP SG12S16, allele G of the SNPs SG12S21, allele T of the SNP SG12S23, allele A of the SNP SG12S25, allele T of the SNP SG12S26, allele T of the SNP SG12S100, allele T of the SNP SG12S28, allele C of the SNP SG12S143, allele G of the SNP SG12S144, and allele G of the SNP SG12S221. Numerous haplotypes with apparent perfect or near perfect correlation with HapK are described in the aforementioned patent documents.

Though the steady state plasma concentration range of 6 μM to 31 μM is contemplated to be effective, every specific subrange, especially integer and half-integer subranges, is contemplated as an alternative embodiment of the invention. For example, in one variation, the dosing schedule is effective to maintain a steady state DG-031 plasma concentration in a range of 8 μM to 28 μM (2.9 μg/ml to 10.1 μg/ml) in said human.

In another variation, the dosing schedule is effective to maintain a steady state DG-031 plasma concentration in a range of 8 μM to 24 μM (2.9 μg/ml to 8.7 μg/ml) in said human. In another variation, the dosing schedule is effective to maintain a steady state DG-031 plasma concentration in a range of 12 μM to 25 μM (4.3 μg/ml to 9.0 μg/ml) in said human. In still another variation, the dosing schedule is effective to maintain a steady state DG-031 plasma concentration in a range of 9 μM to 28 μM (3.2 μg/ml to 10.1 μg/ml) in said human. Minimum plasma concentrations of 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM and so on, are contemplated for the range. Maximum concentrations of 31 μM, 30.5 μM, 30 μM, 29.5 μM, 29 μM, 28.5 μM, 28 μM, 27.5 μM, 27 μM, 26.5 μM, 26 μM, 25.5 μM, 25 μM, 24.5 μM, 24 M . . . 11.5 μM, 11 μM, 10.5 μM, 10 μM are contemplated for the range.

Preferred doses, formulations, and dosing schedules maintain a therapeutically effective plasma concentration of the drug throughout the day, while minimizing the peak concentration to which the person is exposed, because adverse effects and events of drugs are often associated with peak concentrations.

In an embodiment of the invention, the doses and dosing schedules of the methods and uses of the invention are effective to maintain a steady state DG-031 plasma concentration of at least 8 μM. Doses and dosing schedules effective to maintain a steady DG-031 plasma concentration of at least 9 μM, at least 9.5 μM, at least 10 μM, at least 10.5 μM or at least 11 μM are also contemplated.

In another embodiment of the invention, the doses and dosing schedule of the methods and uses of the invention are effective to maintain a steady state DG-031 concentration below 30 μM in said human. Dosing schedules effective to maintain a steady state plasma concentration below 29 μM, below 28 μM, below 27 μM, below 26 μM, or below 25 μM in a human are also contemplated.

In a related embodiment, the invention is a method of treating or preventing an inflammatory condition or disease in a human comprising administering doses of DG-031, or a pharmaceutically acceptable salt or ester or prodrug thereof, to the human according to a dosing schedule that is effective to maintain a steady state DG-031 plasma concentration of at least 6 μM; and provide a peak/trough (Cₘₐₓ/Cₘᵢₙ) plasma concentration ratio of less than 5. Preferably, the peak/though plasma concentration ratio is less than 2.0.

In another embodiment, the invention is a use of DG-031, or a pharmaceutically acceptable salt, ester, or prodrug thereof, for the preparation of a medicament for human administration for the treatment or prevention of an inflammatory condition or disease, wherein the medicament is administered to the human in a dose according to a dosing schedule that is effective to maintain a steady state DG-031 plasma concentration of at least 6 μM; and provide a peak/trough (Cₘₐₓ/Cₘᵢₙ) plasma concentration ratio of less than 5. Preferably, the peak/though plasma concentration ratio is less than 2.0.

As described above, variations of the invention involve maintaining a higher minimum plasma concentration, with every specific integer or half-integer concentration specifically contemplated. Also, variations with smaller peak/trough ratios are specifically contemplated, with smaller ratios generally preferred to minimize side effects and minimize drug consumption. For example, in some variations, the doses and dosing schedule are effective to maintain a steady state DG-031 plasma concentration of at least 8 μM (2.9 μg/ml, or at least 9 μM (3.2 μg/ml), or least 10 μM (3.6 μg/ml) or at least 12 μM (4.3 μg/ml). In some variations, the doses and dosing schedule are effective to provide a peak:trough DG-031 plasma concentration ratio of less than 4, or less than 3.5, or less than 3, or less than 2.5, or less than 2.

In preferred variations of the invention, measurable reductions in one or more inflammatory markers in achieved, preferably an inflammatory marker that correlates with a disease state or is predictive of a likelihood of a disease or condition. Thus, in one variation of methods of the invention, the doses and dosing schedule are effective to cause a reduction of serum C-reactive protein (CRP) of at least 20% within two weeks of commencing administration and maintain said reduction with continued administration of doses according to the dosing schedule. More significant reduction, e.g., at least 25%, 28%, 30%, 32%, 35%, 40%, 45%, 50%, or more, is preferred. Faster reduction, e.g., within 10 days or one week of commencing therapy, is preferred. With respect to a single human, percent reduction is measured relative to pre-treatment levels measurable in the human. Pre-treatment levels can be measured at any time before administration of the drug, although inflammatory markers, such as CRP will vary with diet, activity, infection, other medications, and the like. For greater accuracy, two or more pre-treatment measurements from different times can be used to establish the pre-treatment baseline. With respect to evaluating a dose and dosing schedule or a particular sustained or controlled release formulation in a clinical setting or in a population, mean reductions are used.

In another variation, the inflammatory marker is used as a primary measure of the method of the invention. Thus, in another embodiment, the invention is a method of treating or preventing an inflammatory condition or disease in a human comprising administering doses of DG-031, or a pharmaceutically acceptable salt or ester or prodrug thereof, according to a dosing schedule that is effective to cause a reduction of serum C-reactive protein (CRP) of at least 20% within two weeks of commencing administration, and maintain said reduction with continued administration of doses according to the dosing schedule. As described above, more significant reduction and faster reduction is preferred.

Myeloperoxidase is another preferred inflammatory marker for use as a primary or secondary measure of a method of the invention. Thus, in another variation of methods of the invention, the doses and dosing schedule are effective to achieve a reduction of serum myeloperoxidase (MPO) of at least 30% within one week of commencing administration and maintain said reduction with continued administration of doses according to the dosing schedule. More significant reductions, e.g., of 35% or 40%, are preferred. As described below, measurement of stimulated MPO production, obtained by treating a whole blood sample with a calcium ionophore, such as ionomycin, are contemplated for monitoring drug effects.

In some variations, the materials and methods/uses of the invention are selected to maximize both serum/plasma LTB4 reduction and maximize stimulated whole blood LTB4 reduction. In an initial study (Example 8), both values were further reduced after four weeks of administration in a 500 mg/BID (1000 mg/day total) dosing regimen, compared to a 500 mg/TID (15000 mg total) dosing regimen.

In another variation of methods of the invention, the doses and dosing schedule are effective to achieve a reduction of leukotriene B4 (LTB4) or leukotriene A4 (LTA4)) of at least 20% within one week of commencing administration, and maintain said reduction with continued administration of doses according to the dosing schedule. Greater reductions (e.g., 25%, 30%, 35%, 40%, or more, and faster reductions, e.g., within 5 days or 4 days or 3 days, is preferred. More preferred is a reduction to the baseline LTB4 level in control patients, e.g., 30% to 35% reduction. Measurements of stimulated LTB4 production obtained by treating a whole blood sample with a calcium ionophore, such as ionomycin, are utilized for monitoring efficacy. In addition, LTB4 may be measured in serum or plasma.

In another embodiment, the invention provides for any of the preceding methods or uses to further comprise a step of measuring at least one inflammatory marker in a sample from the human to monitor efficacy of the therapy, wherein a reduction in the inflammatory marker compared to pre-treatment levels is indicative of efficacy. The invention provides for measuring the at least one inflammatory marker at least annually during treatment. The invention also provides for measuring the at least one inflammatory marker within 45 days of beginning the administering. The invention contemplates that the at least one inflammatory marker is a MPO or a leukotriene, such as LTB4.

In some variations, the materials and methods/uses of the invention are selected to minimize undesirable side effects while still meeting therapeutic objectives set forth herein. Undesirable side effects include elevations of serum LDL (e.g., LDL-C), adverse indicators of liver function, increases in serum creatinine (or other potential indicators of adverse affects on renal function), and increases in creatine kinase.

The invention provides methods and uses of the invention wherein the inflammatory disease or condition is a cardiovascular disease or condition. Exemplary disease or conditions for therapeutic or prophylactic therapy include cardiovascular disease, or more particularly, arthrosclerosis, arteriosclerosis, or PAOD; and patients at increased risk for myocardial infarction or stroke due to family history, medical history, behavior (e.g., smoking), or genetic predisposition. Such family or medical history risk factor include diabetes; hypertension; hypercholesterolemia; elevated triglycerides; elevated lp(a); obesity; ankle/brachial index (ABI) less than 0.9; a past or current smoker; transient ischemic attack; transient monocular blindness; carotid endarterectomy; asymptomatic carotid stenosis; claudication; limb ischemia leading to gangrene, ulceration or amputation; a vascular or peripheral artery revascularization graft; increased serum LDL cholesterol and/or decreased HDL cholesterol; serum total cholesterol >200 mg/dl, increased leukotriene synthesis; and/or at least one previous myocardial infarction, ACS, stable angina, previous transient ischemic attack, transient monocular blindness, or stroke, asymptomatic carotid stenosis or carotid endarterectomy, atherosclerosis, requires treatment for restoration of coronary artery blood flow (e.g., angioplasty, stent, revascularization procedure).

In one variation, the doses are in a range of 693-1385 micromoles of the DG-031 (250-500 mg) or the salt or ester thereof, and wherein the dosing schedule is 2 times per day. Every integer dose within the range, including 300 mg, 350 mg, 375 mg, 400 mg, 450 mg, 475 mg, is specifically contemplated. It will be understood that a molar quantity of DG-031 will not necessarily weigh the same as the same molar quantity of one of its salts, esters, or prodrugs. The molecular weight of DG-031 is 361 gram/mole.

In some variations, the doses are in a range of 342-1385 micromoles of the DG-031 (125-500 mg) or the salt or ester thereof, and wherein the dosing schedule is 3-4 times per day. More preferably, the doses are in a range of 485-1108 micromoles of the DG-031 (175-400 mg) or the salt or ester thereof. In another variation, the doses are In a range of 554-831 micromoles of the DG-031 (200-300 mg) or the salt or ester thereof, and the dosing schedule is three times per day (TID).

In another variation, the doses are in a range of 1039-1385 micromoles of the DG-031 (375-500 mg) or the salt or ester thereof, and wherein the dosing schedule is 2 times per day. More preferably, the dose is 1385 micromoles of the DG-031 (500 mg) or the salt or ester thereof 2 times per day (BID).

In one preferred embodiment of this dosing schedule, the doses are 693 or 1385 micromoles of the DG-031 (250 or 500 mg) or the salt or ester thereof. In one variation, the doses are formulated for oral administration and administered orally. An exemplary formulation for use in the method of the invention is a solid tablet that is orally administered and that consists essentially of 250 mg of DG-031, 40 mg of corn starch, 96.24 mg of microcrystalline cellulose, 1.24 mg, 10 mg providone 25 [poly(1-vinyl-2-pyrrolidinone 25], 2.52 mg magnesium stearate and purified water and further comprising a film coating consisting essentially of 6 mg methylhydroxypropylcellulose, 1.5 mg polyethylene glycol 4000, 2.5 mg titanium oxide and purified water. For a 500 mg dose, two tablets are consumed.

In still another variation of the methods of the invention, a controlled or sustained release formulation is administered. Such formulations are convenient for the patient because fewer doses are required, and patient compliance generally improves. Thus, in one embodiment, the dose is administered in a sustained release dosage form and the dosing schedule is twice per day (BID). In another embodiment, the dose is administered in a sustained release dosage form and the dosing schedule is once per day (QD). Exemplary doses for sustained release formulations are in a range of 693-2770 micromoles of the DG-031 (250-1000 mg) or the salt or ester thereof; or in a range of 1039-2076 micromoles of the DG-031 (375-750 mg) or the salt or ester thereof. Another exemplary dose for sustained release formulation is 1385 micromoles of the DG-031 (500 mg) or the salt or ester thereof administered twice a day (BID). A further exemplary dose for sustained release formulation is 2770 micromoles of the DG-031 (1000 mg) or the salt or ester thereof administered once a day (QD).

In another embodiment, the invention is a method of treating or preventing an inflammatory condition or disease in a human comprising administering an initial dose of DG-031, or a pharmaceutically acceptable salt or ester or prodrug thereof according to any of the variation of the methods already described, continuing administering doses of DG-031 according to the initial dosing schedule for a time effective to cause a reduction of leukotriene B4 (LTB4) of at least 30% or at least 35%, and administering a maintenance dose of DG-031, or a pharmaceutically acceptable salt or ester or prodrug thereof, that is less than the initial dose of DG-031 administered. The LTB4 levels may be measured using the ionophore-stimulation assay. A maintenance dose of DG-031 is a dose that effectively maintains a therapeutic benefit such as a measurable reduction in LTB4. A maintenance dosing schedule is a dosing schedule that effectively maintains a therapeutic benefit of the initial dosing schedule that has a reduced quantity of DG-031 per dose and/or a reduced frequency of administration. Preferably, administration of the initial dose is continued for 2 weeks. Exemplary maintenance doses of DG-031 and maintenance dosing schedules are 693 micromoles of the DG-031 (250 mg) or the salt or ester thereof, and the maintenance dosing schedule is two times per day or once per day. Another exemplary maintenance dose is a total daily administered according to the maintenance doses and dosing schedule is at least 25% less than the total daily administration according to the initial doses and dosing schedule.

In another embodiment, the invention is a method of treating or preventing an inflammatory condition or disease in a human comprising administering an initial dose of DG-031, or a pharmaceutically acceptable salt or ester or prodrug thereof according to any of the variation of the methods already described, continuing administering an initial doses of DG-031 according to the initial dosing schedule for a time effective to cause a reduction of serum C-reactive protein (CRP) of at least 20%, and administering a maintenance dose of DG-031, or a pharmaceutically acceptable salt or ester or prodrug thereof, that is less than the initial dose of DG-031 administered. A maintenance dose of DG-031 is a dose that effectively maintains a therapeutic benefit such as a measurable reduction in serum CRP. A maintenance dosing schedule is a dosing schedule that effectively maintains a therapeutic benefit of the initial dosing schedule that has a reduced quantity of DG-031 per dose and/or a reduced frequency of administration. Preferably, administration of the initial dose is continued for 2 weeks. Exemplary maintenance doses of DG-031 and maintenance dosing schedules are 693 micromoles of the DG-031 (250 mg) or the salt or ester thereof, and the maintenance dosing schedule is two times per day or once per day. Another exemplary maintenance dose is a total daily administered according to the maintenance doses and dosing schedule is at least 25% less than the total daily administration according to the initial doses and dosing schedule.

The invention also provides for any of the preceding methods or uses comprising a step, prior to the administering step, of selecting a human at risk for myocardial infarction to receive the doses of DG-031. The selecting step comprises determining a level of an inflammatory marker in a human subject and selecting a subject with an elevated measurement of the marker. The inflammatory markers for selection include CRP, MPO and leukotriene, preferably LTB4.

In one variation, the selecting step comprises selecting a human who has suffered at least one myocardial infarction. The invention also provides for selecting for administration a human with cardiovascular disease, such as arthrosclerosis, PAOD, myocardial infraction or stroke.

In one variation, the selecting step comprises selecting a human female. In another variation, the selecting step comprises selecting a human that is at least 40 years old, or at least 50 years old, or at least 60 years old, or at least 65 years old.

In another variation, the selecting step comprises selecting a human with a genetic predisposition to increased risk for myocardial infarction. For example, the genetic predisposition comprises presence of a polymorphism or haplotype in the human that correlates with increased risk for MI, wherein the polymorphism or haplotype is in a gene selected from the group consisting of FLAP, LTA4-H, and 5-LO.

The invention also provides for the selecting step further comprising determining if a human has a race that includes black African ancestry, and selecting for dosing with DG-031 a human with a race that includes black African ancestry. Further, the selecting step comprises determining if a human has a race that includes European ancestry, and selecting a human with a race that includes European and African ancestry.

Methods of the invention can be practiced by any mode/route of drug administration, including but not limited to oral, transdermal, transmucosal (e.g., sublingual, buccal), intradermal, subcutaneous, intramuscular, intravenous, pulmonary (e.g., nebulizer, metered-dose inhaler) anal, rectal, vaginal, inhalation and intranasal administration. Oral administration, e.g., by a tablet/pill or capsule, is preferred.

Because conditions for which the treatment is indicated may be chronic or progressive, and also because the treatment may be prophylactic, repeated dosing according to the dosing schedule is specifically contemplated, For example, the methods of the invention may be practiced where the administering is performed for at least 30 days, 60 days, 90 days, 120 days, 180 days, 1 year, 2 years, 3 years, or longer, e.g., for the duration of a person's life.

Another embodiment of the invention is a composition comprising a sustained or controlled release formulation of a therapeutic agent described herein. Exemplary sustained or controlled release formulations are those which permit practicing methods of the invention via a dosing schedule of two or fewer doses per day, preferably oral doses.

Thus, one embodiment of the invention is a controlled or sustained release formulation for oral administration to a human comprising DG-031, or a salt or ester or prodrug thereof, in an amount effective to provide a mean minimum plasma concentration (Cₘₐₓ) of DG-031 in the range of 6 μM to 15 μM (2.2 μg/ml to 5.4 μg/ml) and a mean maximum plasma concentration of DG-031 in the range of 10 μM to 31 μM (3.6 μg/ml to 11.2 μg/ml) after repeated oral administration every 12 hours through steady state conditions. A “mean plasma concentration” is calculated as the Plasma Concentration vs. Time Curve (AUC) divided by the time period over which the samples are taken (e.g., AUC(0-24 hrs./24). The mean maximum plasma concentration refers to the maximum concentration on the Plasma vs. Time curve, plotted as average values from a representative number of human subjects. The mean minimum plasma concentration refers to the minimum concentration on the same curve. The human subjects used for the plot are generally adults. However, for a condition affecting a particular subpopulation, the representative sample preferably comprises people with the condition. As an optional refinement, sex-specific or race-specific dosages are made, in which case the mean values are obtained from people of the same sex or race.

As described above with respect to methods of the invention, all integer and half-integer subranges of the maximum and minimums recited above are specifically contemplated as embodiments of the invention. For example, in one variation, the invention is a controlled release formulations that provides a mean minimum plasma concentration (Cₘₐₓ) of DG-031 in the range of 8 μM to 15 μM (2.9 μg/ml to 5.4 μg/ml) or in the range of 10 μM to 15 μM (3.6 μg/ml to 5.4 μg/ml) or in the range of 11 μM to 15 μM (4.0 μg/ml to 5.4 μg/ml) after repeated oral administration every 12 hours through steady state conditions. In another embodiment of the invention, the invention provides for a controlled release formulation that is effective to provide a mean maximum plasma concentration of DG-031 in the range of 15 μM to 31 μM (5.4 μg/ml to 11.2 μg/ml) or in the range of 17 μM to 31 μM (6.1 μg/ml to 11.2 μg/ml) or in a range of 17 μM to 28 μM (6.1 μg/ml to 10.0 μg/ml) or 17 μM to 25 μM (6.1 μg/ml to 9.0 μg/ml) after repeated oral administration every 12 hours through steady state conditions.

In a variation of the invention is a controlled release formulations that provides a mean minimum plasma concentration of DG-031 in the range of 8 μM to 15 μM (2.9 μg/ml to 5.4 μg/ml) and a mean maximum plasma concentration of DG-031 in the range of 20 μM to 28 μM (7.2 μg/ml to 10.1 μg/ml) after repeated oral administration every 12 hours through steady state conditions.

In another variation, the controlled or sustained release formulation provides a mean minimum plasma concentration of DG-031 in the range of 8 μM to 15 μM (2.8 μg/ml to 5.4 μg/ml) and a mean maximum plasma concentration of DG-031 in the range of 20 μM to 24 μM (7.2 μg/ml to 8.7 μg/ml) after repeated oral administration every 12 hours through steady state conditions. In another variation, the controlled release formulation provides a mean minimum plasma concentration of DG-031 in the range of 9 μM to 15 μM (3.2 μg/ml to 5.4 μg/ml) and a mean maximum plasma concentration of DG-031 in the range of 20 μM to 28 μM (7.2 μg/ml to 10.1 μg/ml) after repeated oral administration every 12 hours through steady state conditions.

In still another variation, the controlled or sustained release formulation provides a mean minimum plasma concentration of DG-031 in the range of 12 μM to 17 μM (4.3 μg/ml to 6.1 μg/ml) and a mean maximum plasma concentration of DG-031 in the range of 22 μM to 25 μM) after repeated oral administration every 12 hours through steady state conditions.

As described above with respect to methods of the invention, controlled or sustained release formulations preferably are formulated to minimize the difference between plasma concentration peaks and troughs over time. Thus, in one preferred variation, a controlled release formulation such as described herein is formulated such that the ratio of the mean maximum plasma concentration (peak) and the mean minimum plasma concentration (trough) of DG-031 after repeated oral administration every 12 hours through steady state conditions is less than 5, or more preferably less than 4.5, or less than 4, or less than 3.5, or less than 3, or less than 2.5 or less than 2

An exemplary 12 hour controlled or sustained release formulation contains from 693 to 1385 micromoles of DG-031 (250 mg to 500 mg) or the salt or ester thereof. More preferably, it contains 831 to 1108 micromoles of DG-031 (300 mg to 400 mg) or the salt or ester thereof. Still more preferably, it contains from 970 to 1039 micromoles of DG-031 (350 mg to 375 mg) or the salt or ester thereof. Other preferable controlled release formulations contain contains from 2077 to 2770 micromoles of DG-031 (750 mg to 1000 mg) or the salt or ester thereof or 2770 to 4155 micromoles of DG-031 (1000 mg to 1500 mg) or the salt or ester thereof.

An exemplary 12 hour controlled or sustained release formulation is characterized by a mean maximum plasma concentration of DG-031 that is detectable 4 to 6 hours after administration. Another exemplary 12 hour controlled sustained release formulation is characterized by a mean minimum plasma concentration of DG-031 that is detectable 10 to 12 hours after administration. The controlled release formulations also can be characterized by their effects on inflammatory markers. For example, an exemplary 12 hour formulation preferably decreases LTB₄ production in the human within 4 to 6 hours after administration.

In another embodiment, the invention is a controlled release formulation for oral administration to a human comprising DG-031, or a salt or ester or prodrug thereof, in an amount effective to provide a mean minimum plasma concentration of DG-031 from 6 μM to 15 μM (2.2 μg/ml to 5.4 μg/ml) and a mean maximum plasma concentration of DG-031 from 20 μM to 31 μM (7.22 μg/ml to 11.2 μg/ml) after repeated administration every 24 hours through steady state conditions. As described above, every integer and half-integer sub-range concentration is specifically contemplated for both the maximum and minimum.

For example, in one variation, the invention is a controlled release formulation that provides a mean minimum plasma concentration (Cₘₐₓ) of DG-031 in the range of 8 μM to 15 μM (2.9 μg/ml to 5.4 μg/ml) or in the range of 10 μM to 15 μM (3.6 μg/ml to 5.4 μg/ml) or in the range of 15 μM to 31 μM (2.9 μg/ml to 11 μg/ml) or in the range of 17 μM to 31 μM (6.1 μg/ml to 11.2 μg/ml) or in the range of 17 μM to 28 μM (6.1 μg/ml to 10.0 μg/ml) after repeated oral administration every 24 hours through steady state conditions.

Thus, in one variation, the controlled release formulation provides a mean minimum plasma concentration of DG-031 in the range of 8 μM to 15 μM (2.8 μg/ml to 5.4 μg/ml) and a mean maximum plasma concentration of DG-031 in the range of 20 μM to 28 μM (7.2 μg/ml to 8.7 μg/ml) after repeated oral administration every 24 hours through steady state conditions. In another variation, it provides a mean minimum plasma concentration of DG-031 in the range of 8 μM to 15 μM (2.8 μg/ml to 5.4 μg/ml) and a mean maximum plasma concentration of DG-031 in the range of 20 μM to 24 μM (7.2 μg/ml to 8.7 μg/ml) after repeated oral administration every 24 hours through steady state conditions. In still another variation, it provides a mean minimum plasma concentration of DG-031 in the range of 9 μM to 15 μM (3.2 μg/ml to 5.4 μg/ml) and a mean maximum plasma concentration of DG-031 in the range of 20 μM to 28 μM (7.2 μg/ml to 10.1 μg/ml) after repeated oral administration every 24 hours through steady state conditions. In another variation, the controlled release formulation provides a mean minimum plasma concentration of DG-031 in the range of 12 μM to 17 μM (4.3 μg/ml to 6.1 μg/ml) and a mean maximum plasma concentration of DG-031 in the range of 22 μM to 25 μM (7.9 μg/ml to 9.0 μg/ml) after repeated oral administration every 24 hours through steady state conditions.

Preferred ratios of the mean maximum plasma concentration (peak) and the mean minimum plasma concentration (trough) of DG-031 for the 24 formulation are as described above for the 12 hour formulation. An exemplary dose range for a 24 hour formulation is from 1108 to 2770 micromoles of DG-031 (400 mg to 1000 mg of DG-031) or the salt or ester thereof; or more preferably from 1662 to 2355 micromoles of DG-031 (600 mg to 850 mg of DG-031) or the salt or ester thereof; or from 1939 to 2216 micromoles of DG-031 (700 mg to 800 mg of DG-031) or the salt or ester thereof. Another exemplary dose range for a 24 hour formulation is from 2077 to 2770 micromoles of DG-031 (750 mg to 1000 mg of DG-031) or the salt or ester thereof. Another exemplary dose range for a 24 hour formulation is from 2770 to 4155 micromoles of DG-031 (1000 mg to 1500 mg of DG-031) or the salt or ester thereof.

In one variation, the 24 hour controlled release formulation results in a mean maximum plasma concentration of DG-031 that is detectable 10 to 12 hours after administration, and a mean minimum plasma concentration of DG-031 that is detectable 20 to 24 hours after administration. The formulation preferably causes a decrease in LTB₄ production in a human within 4 to 12 hours after administration.

Other markers for characterizing controlled release formulations of the invention include LTB4, MPO and CRP. In one embodiment, the controlled release DG-031 formulation causes a decrease in stimulated LTB4 levels in a human within 4 hours after administration. In another embodiment, the controlled release DG-031 formulation causes a decrease in serum MPO levels in a calcium ionophore-stimulated sample of blood from the human within 6 hours after administration. In a further embodiment, the controlled release DG-031 formulation causes a decreases serum CRP levels in the human within 1 week of beginning daily administration.

As described above, formulations for all modes of administration are contemplated, but oral is preferred. Thus, a formulation that is a solid tablet or capsule is preferred.

In some formulations, the solid tablet comprises a film coating. The film coating is a solid formulation that acts as a controlled or sustained release matrix which results in the prolonged or extended release of DG-031. Preferred film coatings reduce dissolution of the tablet in stomach acid with a pH less than 5.0. Other preferred film coatings reduce the percent dissolution of the tablet in stomach acid with a pH less than 5.0, and wherein the film coating allows for tablet dissolution at a pH greater than 6.0. Exemplary film coatings of the invention comprises Eudragit L 100, Eudragit S100, Eudragit L 100-55, Colorcon Surlease, or FMC Aquacoat CPD.

In another embodiment, the invention provides for a controlled or sustained release oral dosage formulation comprising DG-031 or a salt thereof in an amount as described above and an effective amount of a controlled or sustained release matrix selected from the group consisting of methylhydroxypropylcellulose, hypomellose phthalate polymer, ethylcellulose, polymethacrylate, hydroxypropyl methylcellulose acetate succinate, cellulose acetate phthalate (CAP) polymer and acrylic resin and further optionally comprising a pharmaceutically acceptable diluent.

In a further embodiment, the invention provides for a use DG-031, or a pharmaceutically acceptable salt or ester or prodrug thereof, for the preparation of a medicament for human administration for the treatment or prevention of an inflammatory condition or disease, wherein the medicament is to be administered at an initial dose according to an initial dosing schedule that is effective to maintain a steady state DG-031 plasma concentration in a range of 6 μM to 31 μM in said human, wherein the initial dose and dosing schedule are continued for a time effective to cause a reduction of LTB4 of at least 30%, and wherein the medicament is then administered at a maintenance dose of DG-031, or a pharmaceutically acceptable salt or ester or prodrug thereof, according to a maintenance dosing schedule after the reduction in LTB4, wherein the maintenance dose of DG-031 and the maintenance dosing schedule are effective to maintain a reduction of serum LTB4 of at least 30%.

In a variation of the invention, the total daily administration according to the maintenance doses and dosing schedule of the preceding use is at least 25% less than the total daily administration according to the initial doses and dosing schedule. The invention contemplates maintenance doses of 693 micromoles of the DG-031 (250 mg) or the salt or ester or prodrug thereof, and the maintenance dosing schedule is two times per day, or maintenance doses are 693 micromoles of the DG-031 (250 mg) or the salt or ester or prodrug thereof, and the maintenance dosing schedule is once per day. The invention also provides for any of the preceding methods or uses wherein the administering of the initial doses of DG-031 continues for 2 weeks.

Still another variation of the invention is a kit that comprises doses of a medicament as described herein packaged to facilitate administration according to the invention. For example, the package may contain separated doses marked as individual doses and/or marked for once daily, twice daily, or three times daily administration.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. With respect to aspects described as a range, all subranges and individual values are specifically contemplated.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### BRIEF DESCRIPTION OF THE DRAWING

FIG. 1 is a schematic depicting the cross-over design of the randomized, cross-over clinical study (Study No. DG-031-201) and the flow of participants through the stages of the study. For the cross-over study, the same subjects take drug and placebo in 4 week blocks.

FIG. 2 depicts the carry-over effect for CRP (on log-scale). Patients were exposed to drug beginning at visit 2 and continued therapy until visit 4 over a period of 4 weeks. Therapy with DG-031 was ended at visit 4. At visits 5, 6 and 7 (2-4 weeks after the end of therapy with DG-031), there was a continuing and persistent reduction in CRP.

FIG. 3 depicts the change in CRP levels for subjects of clinical study DG-031-203 (see Example 4) and the subjects are divided by median CRP level at entry into the study. This figure illustrates the benefit of TID dosing vs. BID dosing for reduction of CRP.

FIG. 4 depicts, in panel A, the expected DG-031 steady-state concentrations with three times per day dosing (TID) with 250 mg per dose (from clinical study DG-031-203). Panel B depicts the expected DG-031 steady-state concentrations with twice per day dosing (BID) with 375 mg from clinical study DG-031-203.

FIG. 5 depicts the concentration-time profile of the mean steady-state DG-031 plasma concentration after administration of 500 mg DG-031 BID compared to individuals' observed concentrations after administration. In one embodiment, the controlled release DG-031 formulation causes a decrease in serum MPO levels in a human within 6 hours after administration. The concentrations in this figure are depicted as μg/ml and the time plotted spans 6 hours after two weeks of administration of DG-031 at 500 mg BID.

FIG. 6 demonstrates that DG-031 500 mg BID dosing schedule provides the target reduction in stimulated LTB4 levels after 4 weeks of administration.

FIG. 7 depicts the DG-031 PK based on the population average PK model described in Example 9 that was developed from clinical studies DG-031-201 and DG-031-203. This figure displays the PK for the following dosing schedules: 250 TID, 375 BID, 375 BID and 500 BID. The concentrations in this figure are depicted as μg/ml and the time is plotted spans 24 hours after 2 days of administration of DG-031 with the various dosing schedules.

FIG. 8 depicts the concentration-time profile of the mean steady-state DG-031 plasma concentration after administration of 500 mg DG-031 BID and 500 TID. The concentrations in this figure are depicted as μg/ml and the time plotted spans 6 hours after two weeks of administration of DG-031 at 500 mg BID or 500 mg TID.

### DETAILED DESCRIPTION

The use of leukotriene inhibitors to treat cardiovascular diseases is described in International Patent Application Nos. PCT/US03/32805, PCT/US04/30582 and PCT/US05/00312, incorporated herein by reference in their entirety. One preferred class of compounds for use in such materials and methods are FLAP inhibitors described in U.S. Pat. Nos. 4,970,215 and 5,693,650, also incorporated herein by reference in their entirety. A preferred compound for use in such therapeutic materials and methods is DG-031 (also known as Bay x-1005), an orally active inhibitor of the synthesis of leukotrienes B4 and C4 through inactivation of FLAP. DG-031 is a substituted 4-(quinolin-2-YL-methoxy)phenyl-acetic acid derivative. Clinical studies have demonstrated DG-031 was safe and well tolerated in healthy volunteers at a total daily dose of 100 mg, 200 mg, 250 mg, 300 mg, 500 mg, 750 mg, and 1000 mg DG-031 administered for less than 14 days. In addition, total daily doses of 500 mg and 1000 mg DG-031 administered for 14-42 days were safe and well tolerated in healthy volunteers. (See, Dahlen et al. Thorax 523: 348-354, 1997; Hamilton et al., Thorax 52: 348-54, 1997).

In clinical studies in asthma patients, total daily dose of 250 mg, 500 mg, 750 mg and 1000 mg were safe and well tolerated when administered for 14 days. Total daily doses of 100 mg, 200 mg, 250 mg, 500 mg and 1000 mg of DG-031 were safe and well tolerated after being administered for 14-42 days. A total daily dose of 500 mg administered for greater than 45-365 days was also safe and well tolerated in asthma patients.

In clinical studies in patients with coronary artery disease (CAD), total daily doses of 375 mg and 750 mg were safe and well tolerated after being administered for less than 8 days. In addition, total daily doses of 250 mg, 500 mg, and 750 mg of DG-031 were safe and well tolerated in CAD patients after being administered for 128 days. Previous clinical studies administered the dosages of DG-031 either in a single dose (QD) or twice a day (BID)

Improved dosing materials and methods for therapy with DG-031 and related compounds are described herein.

The following doses of DG-031 in CAD patients were tested: 250 mg QD, 375 mg QD, 250 mg BID, 375 mg BID and 250 mg TID, and these studies demonstrated that all doses were safe and well tolerated. A total daily dose of 750 mg, dosed at 250 mg TID, provides a preferable PK/PD relationship (inhibition of LTB₄) and efficacy in terms of lowering other biomarkers of MI risk (i.e. MPO and CRP). In addition, a total daily dose of 1000 mg, dosed at 500 mg BID, also provides a preferable PK and PD time course (inhibition of LTB₄) and efficacy in terms of lowering other biomarkers of MI risk (i.e. MPO). Patient compliance is an important consideration when designing a dosing schedule and patients are more likely to comply with a twice a day dosing (BID) than three times a day doing (TID). A similar PD time course was observed with 250 mg TID dosing and 500 mg BID dosing, but the 500 mg BID dosing schedule may be preferred in view of patient compliance.

In particular, the studies in Examples 1-4 demonstrate that plasma concentrations at levels of 2-3 μg/ml (Cₘᵢₙ) provide consistent reduction of capacity for LTB₄ production measured ex vivo, dose-dependent effects of DG-031 on serum CRP, dose-dependent effects of DG-031 on plasma MPO levels and acceptable safety and tolerability profile.

### Description of Chemical Compounds

DG-031 and related compounds are described in detail in U.S. Pat. No. 4,970,215 (Mohrs, et al.), incorporated herein by reference. Chemical syntheses are described in U.S. Pat. No. 5,693,650, also incorporated by reference.

The compound can be a substituted 4-(quinolin-2-61-methoxy)phenylacetic acid derivative represented by the following formula:

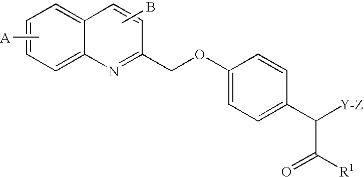

or pharmaceutically acceptable salt thereof, wherein R¹ represents a group of the formula:

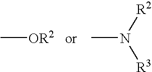

R² and R³ are identical or different and represent hydrogen, lower alkyl, phenyl, benzyl or a group of the formula:

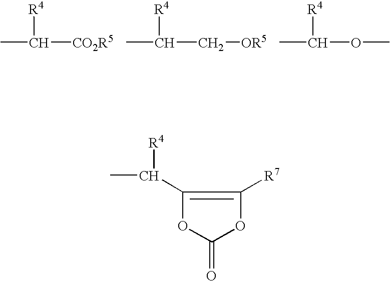

R⁴ represents hydrogen, lower alkyl, phenyl or benzyl, which can optionally be substituted by hydroxyl, carboxyl, lower alkoxycarbonyl, lower alkylthio, heteroaryl or carbamoyl, R⁵ represents hydrogen, lower alkyl, phenyl or benzyl, R⁶ represents a group of the formula —COR⁵ or —CO²R⁵, R⁷ represents hydrogen, lower alkyl or phenyl, Y represents a group of the formula:

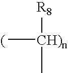

wherein R⁸ represents hydrogen, lower alkyl or phenyl and n denotes a number of 0 to 5, Z represents norbornyl, or represents a group of the formula:

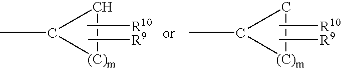

wherein R⁹ and R¹⁰ are identical or different and denote hydrogen, lower alkyl or phenyl, or R⁹ and R¹⁰ can together form a saturated carbocyclic ring having up to 6 carbon atoms and m denotes a number from 1 to 6, and A and B are identical or different and denote hydrogen, lower alkyl or halogen, or a pharmaceutically acceptable salt thereof.

Preferably the compounds are selected from the group consisting of: 2-[4-(quinolin-2-yl-methoxy)phenyl]-2-cyclopentylacetic acid, 2-[4-(quinolin-2-yl-methoxy)phenyl]-2-cyclohexylacetic acid, and 2-[4-(quinolin-2-yl-methoxy)phenyl]-2-cycloheptylacetic acid, (+)-enantiomer of 2-[4-(quinolin-2-yl-methoxy)phenyl]-2-cyclopentylacetic acid, (−)-enantiomer of 2-[4-(quinolin-2-yl-methoxy)phenyl]-2-cyclopentylacetic acid and pharmaceutically acceptable salts thereof. See U.S. Pat. No. 4,970,215, incorporated herein by reference.

A preferred compound is (R)-(+)-alpha-cyclopentyl-4-(2-quinolinylmethoxy)-Benzeneacetic acid, also known as Bay-X1005 and DG-031.

The invention also contemplates physiologically acceptable salts of the compounds of the invention, such as salts of organic or inorganic bases or acids. Physiologically acceptable salts of the substituted 4-(quinolin-2-yl-methoxy)phenylacetic acids, esters and amides can be salts of the substances according to the invention with mineral acids, carboxylic acids or sulphonic acids. Particularly preferred salts are, for example, those with hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, citric acid, fumaric acid, maleic acid or benzoic acid. Salts in the context of the present invention are furthermore salts of monovalent metals, such as alkali metals and ammonium salts. Sodium, potassium and ammonium salts are preferred.

Physiologically acceptable salts can also be metal salts or ammonium salts of the compounds according to the invention which have a free carboxyl group or a tetrazolyl radical. Particularly preferred salts are, for example sodium potassium, magnesium or calcium salts, as well as ammonium salts, which are derived from ammonia, or organic amines, such as, for example, ethylamine, di- or triethylamine, di- or triethanolamine, dicyclohexylamine, dimethylaminoethanol, glucosamine, arginine, lysine, ethylenediamine or 2-phenylethylamine.

A heterocyclic radical in general is a 5- to 6-membered, saturated, partially unsaturated or unsaturated ring which can contain up to 3 oxygen, sulphur and/or nitrogen atoms as heteroatoms. Preferred rings are 5 and 6-membered rings with one oxygen, sulphur and/or up to 2 nitrogen atoms. Rings which are mentioned as preferred are: thienyl, furyl, pyrrolyl, pyrazolyl, pyridyl, pyrimidyl, pyrazinyl, pyridazinyl, thiazolyl, oxazolyl, imidazolyl, pyrrolidinyl, piperidinyl or piperazinyl.

A 5- to 6-membered saturated heterocyclic radical which can also contain up to 3 oxygen, sulphur and/or nitrogen atoms as heteroatoms is in general piperidyl, morpholinyl, piperazinyl or pyrrolidyl. Morpholinyl is preferred.

A carbocyclic radical in general is a 3- to 7-membered, preferably 5- to 7-membered, saturated hydrocarbon ring. Cyclopentyl, cyclohexyl or cycloheptyl are mentioned as preferred.

A hydroxy-protective group in the context of the abovementioned definition is in general a protective group from the series consisting of: tert-butoxydiphenylsilyl, trimethylsilyl, triethylsilyl, triisopropylsilyl, tert-butyl-dimethylsilyl, tert-butyl-diphenylsilyl, triphenylsilyl, trimethylsilylethoxycarbonyl, benzyl, benzyloxycarbonyl, 2-nitrobenzyl, 4-nitrobenzyl, 2-nitrobenzyloxycarbonyl, 4-nitrobenzyloxycarbonyl, tert-butyloxycarbonyl, allyloxycarbonyl, 4-methoxybenzyl, 4-methoxybenzyloxycarbonyl, formyl, acetyl, trichloroacetyl, 2,2,2-trichloroethoxycarbonyl, 2,4-dimethoxymethyl, 2,4-dimethoxybenzyloxycarbonyl, methylthiomethyl, methoxyethoxymethyl, 2-(trimethylsilyl)ethoxy!methyl, 2-(methylthiomethoxy)ethoxycarbonyl, benzoyl, 4-methylbenzoyl, 4-nitrobenzoyl, 4-fluorobenzoyl, 4-chlorobenzoyl or 4-methoxybenzoyl. Acetyl, benzoyl, benzoyl, or methylbenzyl are preferred.

Amino-protective groups in the context of the invention are the customary amino-protective groups used in peptide chemistry. These include, preferably: benzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 4-nitrobenzyloxycarbonyl, 2-nitrobenzyloxycarbonyl, 2-nitro-4,5-dimethoxybenzyloxycarbonyl, methoxycarbonyl, ethoxycarbonyl, propoxycarbonyl, isopropoxycarbonyl, butoxycarbonyl, isobutoxycarbonyl, tert-butoxycarbonyl, allyloxycarbonyl, vinyloxycarbonyl, 2-nitrobenzyloxycarbonyl, 3,4,5-trimethoxylbenzyloxycarbonyl, cyclohexoxycarbonyl, 1,1-dimethylethoxycarbonyl, adamantylcarbonyl, phthaloyl, 2,2,2-trichloroethoxycarbonyl, 2,2,2-trichloro-tert-butoxycarbonyl, methyloxycarbonyl, phenoxycarbonyl, 4-nitrophenoxycarbonyl, fluorenyl-9-methoxycarbonyl, formyl, acetyl, propionyl, pivaloyl, 2-chloroacetyl, 2-bromoacetyl, 2,2,2-trifluoroacetyl, 2,2,2-trichloroacetyl, benzoyl, 4-chlorobenzoyl, 4-bromobenzoyl, 4-nitrobenzoyl, phthalimido, isovaleroyl or benzyloxymethylene, 4-nitrobenzyl, 2,4-dinitrobenzyl or 4-nitrophenyl.

The compounds according to the invention can be in stereoisomeric forms which either behave as image and mirror image (enantiomers) or do not behave as image and mirror image (diastereomers). The invention relates both to the antipodes and to the racemic forms as well as the diastereomer mixtures. The racemic forms, like the diastereomers, can be resolved into the stereoisomerically uniform constituents in a known manner (compare E. L. Eliel, Stereochemistry of Carbon Compounds, McGraw Hill, 1962).

The formulation of the invention may be made by process known in the art and in particular by the process taught in U.S. Pat. Nos. 4,970,215 and 5,693,650, which are herein incorporated by reference in their entirety.

### Methods of Treatment

The invention provides materials and methods to achieve and maintain steady state plasma concentration within concentration ranges that exhibit the most beneficial effects and reduce drug exposure, thereby reducing the possibility of short term or long term drug side-effects. The invention also provides for methods of administering doses of DG-031 according to a dosing schedule of DG-031 that is effective to achieve and maintain a steady state plasma concentration at a desired concentration.

A desired plasma concentration of DG-031 is a concentration that achieves a desired therapeutic endpoint while minimizing side effects. An exemplary therapeutic endpoint is prophylaxis against myocardial infarction, e.g., reducing the likelihood that a person at risk for MI will incur an MI over a period of time through drug therapy. However, other, more readily measurable criteria can be used as a measure of efficacy. For example, a desired plasma concentration is a concentration that effectively reduces concentrations of measurable leukotrienes or leukotriene metabolites and/or reduces the levels (concentrations) of other inflammatory markers in a human subject (or in a biological sample from the human subject). One preferred dose schedule provides administering doses of DG-031 in a concentration/quantity and at a frequency effective to maintain a steady state plasma concentration between 6 μM to 31 μM (2.2 μg/ml to 11.2 μg/ml) of DG-031. All subranges within this range are specifically contemplated, e.g., 6-30 μM, 6-29 μM, 28 μM, 6-27 μM, 6-25 μM, 6-20 μM, 6-15 μM, 6-12 μM, 6-10 μM, 7-31 μM, 8-31 μM, 7-25 μM, 8-25 μM, 7-20 μM, 12-22 μM, 13-25 μM and so on.

An alternative way to describe a dosing schedule of the invention is with respect to one or more of the measurable biological markers affected by the therapeutic agent. For example, an exemplary dosing schedule of the invention provides administering doses of DG-031 in an amount and at a frequency and in a formulation effective to maintain 30%, 35% or greater reduction in a leukotriene (or leukotriene metabolite) level. Similarly, the invention includes materials and methods for achieving even great measures of efficacy. For example, the invention includes administering DG-031 at a dose or doses according to a dosing schedule effective to reduce a concentration/level of a leukotriene or leukotriene metabolite, such as LTA4 and LTB4, by 25%. 30%, 35% or greater when compared to pre-treatment leukotriene levels, and maintains such a reduction. For example, the dosing schedules are contemplated to maintain a reduction of at least 27%, 30%, 32%, 35%, 40%, 45%, 50%, 55%, 60%, 65%, 70%, 75%, 80%, 85%, 90% or 95% compared to pre-treatment levels. The leukotriene level can be measured in a blood sample after stimulating the sample with a calcium ionophore, as described in Example 3. Biological samples in which to measure leukotriene levels include blood, serum, plasma or urine.

In some variations of the invention, repeated doses of DG-031 are administered to maintain a plasma concentration in a desired steady state range. An exemplary repeated dosing schedule is 250 mg of DG-031 three times a day at equal or nonuniform intervals. Another exemplary repeated dosing schedule is 500 mg of DG-031 two times a day at equal or nonuniform intervals.

In other variations of the invention, a sustained or controlled or sustained release formulation is used to achieve a steady state plasma concentration in a desired range with two administrations per day, or one administration per day, or administration every other day, or every third day, or once per week. One preferred controlled or sustained release formulation contains an amount of DG-031 effective to provide a mean minimum plasma concentration of DG-031 from 5 μM to 10 μM (1.8 μg/ml to 3.6 μg/ml) and a mean maximum plasma concentration of DG-031 from 20 μM to 30 μM (7.2 μg/ml to 10.8 μg/ml) after repeated administration every 12 hours through steady state conditions. Another preferred controlled or sustained release formulation contains an amount of DG-031 effective to provide a mean minimum plasma concentration of DG-031 from 12 μM to 17 μM (4.3 μg/ml to 6.1 μg/ml) and a mean maximum plasma concentration of DG-031 from 22 μM to 25 μM (7.9 μg/ml to 9.0 μg/ml) after repeated administration every 12 hours through steady state conditions.

A preferred controlled or sustained release formulation administers an amount of DG-031 effective to provide a mean minimum from 5 μM to 10 μM (1.8 μg/ml to 3.6 μg/ml) and a mean maximum plasma concentration of DG-031 from 10 μM to 30 μM (3.0 μg/ml to 10.8 μg/ml) after repeated administration every 24 hours through steady state conditions. Another preferred controlled or sustained release formulation contains an amount of DG-031 effective to provide a mean minimum plasma concentration of DG-031 from 12 μM to 17 μM (4.3 μg/ml to 6.1 μg/ml) and a mean maximum plasma concentration of DG-031 from 20 μM to 25 μM (7.2 μg/ml to 9.0 μg/ml) after repeated administration every 24 hours through steady state conditions.

The mean plasma concentrations are calculated by dividing the Area Under the Plasma Level vs. Time Curve (AUC) by the time period over which the samples are taken, e.g. (AUC(0-24 hrs)/24.

The dosing schedules of the invention are contemplated for use in treating (prophylactic and/or therapeutic) inflammatory diseases and cardiovascular diseases associated with FLAP activity or FLAP levels or with other members of the leukotriene pathway, such as LTB4 and LTA4. The invention also provides dosing schedules and formulations to treat inflammatory and cardiovascular disease states.

The DG-031 dosing schedules of the invention include treating inflammatory diseases and cardiovascular diseases associated with leukotriene pathway members such as FLAP, arachidonate 5-lipoxygenase (5-LO), leukotriene A4 hydrolase (LTA4H), leukotriene B4 12-hydroxydehydrogenase (LTB4DH) and potentially also leukotriene C4 synthase (17c45)); receptors and/or binding agents of the enzymes; and receptors for the leukotrienes LTA4, LTB4, LTC4, LTD4, LTE4, Cys LT1, Cys LT2, including leukotriene B4 receptor 1 (BLT1), leukotriene B4 receptor 2 (BLT2), and potentially cysteinyl leukotriene receptor 1 (CysLTR1), and cysteinyl leukotriene receptor 2 (CysLTR2).

The invention also contemplates treating and palliating or preventing inflammatory disease states such as rheumatoid arthritis, psoriatic arthritis, inflammatory arthritis, osteoarthritis, inflammatory joint disease, autoimmune disease including autoimmune vasculitis, multiple sclerosis, lupus, diabetes (e.g., insulin diabetes), inflammatory bowel disease, inflammatory eye disease, transplant rejection, graft vs. host disease, and inflammatory conditions resulting from strain, sprain, cartilage damage, trauma, orthopedic surgery, infection or other disease processes, psoriasis, eczema, allergies, acute or chronic lung injury including interstitial lung disease, acute respiratory disease syndrome, pulmonary hypertension, emphysema, cystic fibrosis, pulmonary fibrosis and asthma, acute and chronic glomerulonephritis, uveitis, endometriosis, cute pancreatitis, chronic fatigue syndrome, fibromyalgia, and Kawasaki's disease, and inflammatory eye disease.

Other diseases to prevent or palliate include cardiovascular disease such as myocardial infarction; transient ischemic attack, transient monocular blindness or stroke, or susceptibility to stroke; methods of treatment for claudication, PAOD or susceptibility to PAOD; methods of treatment for acute coronary syndrome (e.g., unstable angina, non-ST-elevation myocardial infarction (NSTEMI) or ST-elevation myocardial infarction (STEMI)); methods for reducing risk of MI, stroke or PAOD in persons with asymptomatic ankle/brachial index less than 0.9; methods for decreasing risk of a second myocardial infarction or stroke; methods of treatment for atherosclerosis, such as for patients requiring treatment (e.g., angioplasty, stents, revascularization procedure) to restore blood flow in arteries (e.g., coronary, carotid, and/or femoral arteries); methods of treatment for asymptomatic ankle/brachial index of less than 0.9; and/or methods for decreasing leukotriene synthesis (e.g., for treatment of myocardial infarction, stroke or PAOD).

Serum CRP and MPO levels are individually known to be strong predictors of risk for cardiovascular disease such as myocardial infarction. The DG-031 dosing schedules of the invention can also be used to reduce the levels of inflammatory markers, such as CRP and MPO, in a human. Particularly, the invention contemplates carrying out methods of reducing inflammatory markers comprising administering a dose or doses of DG-031 according to a dosing schedule of the invention to a human suffering from an inflammatory disorder, suffering from a cardiovascular disease or at risk for developing a cardiovascular disease.

An increasing body of emerging evidence identifies serum CRP as a marker for cardiovascular morbidity/mortality, and correlates reductions in serum CRP to better clinical outcomes. (See, e.g., Ridker et al., N. Engl. J. Med. 352(1): 20-28 (2005); Nissen et al., N. Engl. J. Med. 352(1): 29-38 (2005); and Pearson et al., Circulation 107: 499-511 (2003).) Serum CRP in excess of 3.0 mg/L is considered high risk; from 1.0 to 3.0 average risk; and below 1 mg/L low risk. (Pearson et al.) Compositions and methods of the invention provide tools for reducing serum CRP. Reductions in CRP can be measured on a concentration basis, where compositions and methods that achieve CRP below 3.0 mg/L are preferred; with still more preferred targets of 2.75 mg/L, 2.5 mg/L, 2.25 mg/L, 2.0 mg/L, 1.75 mg/L, 1.5 mg/L, 1.25 mg/L, 1.0 mg/L, 0.75 mg/L, and 0.5 mg/L. Reductions in CRP also can be measured on a percentage basis, where clinical effectiveness is evaluated as a percentage reduction in CRP in a patient compared to before treatment with a dosing schedule of the invention. Depending on the initial CRP measurement, DG-031 dosing schedules and methods that reduce CRP anywhere from 10%-90% or more are contemplated, e.g., reductions of 10%,15%,20%, 25%, 30%, 40%, 50%, 60%, 65%, 70%, 75%, 80%, or any target in between these values.

MPO also inactivates protease inhibitors and consumes nitric oxide, all of which escalate the inflammatory response (Eiserich et al., Science 296:2391-4, 2002). MPO has been shown to be elevated in patients with documented coronary artery disease (CAD) and within atherosclerotic lesions that are prone to rupture (Zhang et al., JAMA, 286:2136-2142, 2001; Sugiyama et al., Am J Pathol., 158:879-9, 2001). MPO is also elevated in patients with chest pain and predictive of subsequent cardiovascular events at 3 and 6 months (Brennan N Engl J Med., 349:1595-604, 2003). Reductions in MPO can be measured on a percentage basis, where clinical effectiveness is evaluated as a percentage reduction in MPO in a patient compared to prior treatment with a dosing schedule of the invention. Depending on the initial MPO measurement, DG-031 dosing schedules and methods that reduce MPO anywhere from 10%-90% or more are contemplated, e.g., reductions of 10%, 15%, 20%, 25%, 30%, 40%, 50%, 60%, 65%, 70%, 75%, 80%, or any target in between these values.

The identification of a human in need of treatment for CRP or MPO reduction can be based on a variety of factors described herein, including genetic factors, CRP measurements, MPO measurements and measurements of other inflammatory markers, and measurements of non-genetic and non-inflammatory markers for risk of cardiovascular disease. In one variation, the method includes selecting for the administering step a human subject at risk for a disease or condition selected from the group consisting of myocardial infarction, acute coronary syndrome, stroke, or peripheral arterial occlusive disease.

In still another variation, the monitoring of markers of inflammation is used to adjust the dosing schedule for those individuals suffering from inflammatory diseases and those individuals at risk of or suffering from cardiovascular disease. For example, dose or dosing of a DG-031 is increased if serum CRP, and/or MPO and/or serum or urinary leukotriene measurements do not decrease to a target level, such as a level equivalent to the bottom 50 percentile, 40 percentile, 30 percentile, 20 percentile, 10 percentile, 1 percentile of a population, or other target percentile in between these exemplary targets. As described above, monitoring also can be used to adjust dosing to achieve a target level of serum CRP or MPO level, or to achieve a target percentage reduction in CRP or MPO for a particular human subject.

### Target Populations

In an embodiment of the invention, the dosing schedule of DG-031 of the invention may be administered to a human at risk for cardiovascular disease, such as MI, ACS, stroke or PAOD. Increased risk for MI, ACS, stroke or PAOD in individuals may have increased production of leukotrienes (e.g., LTA4, LTB4, LTC4, LTD4, LTE4,). For example, the increased production of leukotrienes may be in the arterial vessel wall or in bone-marrow derived inflammatory cells within the blood and/or arterial vessel wall.

In another embodiment, the DG-031 dosing schedule is administered to humans having elevated levels of other inflammatory markers. An “elevated inflammatory marker,” as used herein, is the presence of an amount of an inflammatory marker that is greater, by an amount that is statistically significant, than the amount that is typically found in control individual(s) or by comparison of disease risk in a population associated with the lowest band of measurement (e.g., below the mean or median, the lowest quartile or the lowest quintile) compared to higher bands of measurement (e.g., above the mean or median, the second, third or fourth quartile; the second, third, fourth or fifth quintile). An “inflammatory marker” refers to a molecule that is indicative of the presence of inflammation in an individual, for example, an end product of the leukotriene pathway, such as LTB4, C-reactive protein (CRP), serum sCD40L, serum amyloid A, fibrinogen, a leukotriene, a leukotriene metabolite, interleukin-6, tissue necrosis factor-alpha, a soluble vascular cell adhesion molecule (sVCAM), a soluble intervascular adhesion molecule (sICAM), E-selectin, matrix metalloprotease type-1, matrix metalloprotease type-2, matrix metalloprotease type-3, matrix metalloprotease type-9, myeloperoxidase (MPO), and N-tyrosine.

The invention also provides for administering the DG-031 dosing schedule of the invention to a human having a genetic risk factor for CAD or MI. Human subjects having a polymorphism or haplotype in the FLAP gene or LTA4H gene that is associated with risk of MI or CAD. Exemplary FLAP haplotypes that are associated with risk of MI or CAD are HapA, HapB, HapC, which are described in the aforementioned patent documents. Exemplary LTA4H haplotypes that are associated with risk of MI or CAD are HapK, HapL and HapQ, and surrogate haplotypes thereof, which are described in detail in the aforementioned patent documents.

The invention further provides for administering the DG-031 dosing schedule to humans having at least one family or medical history risk factor such as diabetes; hypertension; hypercholesterolemia; elevated triglycerides; elevated lp(a); obesity; ankle/brachial index (ABI) less than 0.9; a past or current smoker; transient ischemic attack; transient monocular blindness; carotid endarterectomy; asymptomatic carotid stenosis; claudication; limb ischemia leading to gangrene, ulceration or amputation; a vascular or peripheral artery revascularization graft; increased serum LDL cholesterol and/or decreased HDL cholesterol; serum total cholesterol >200 mg/dl, increased leukotriene synthesis; and/or at least one previous myocardial infarction, ACS, stable angina, previous transient ischemic attack, transient monocular blindness, or stroke, asymptomatic carotid stenosis or carotid endarterectomy, atherosclerosis, requires treatment for restoration of coronary artery blood flow (e.g., angioplasty, stent, revascularization procedure).

The invention also provides for administering the DG-031 dosing schedule to women of any age, men and women over the age of 40 years, such as those over the age of 45, 50, 55, 60, 65, 70, 75 and 80 and human subjects that have a race that includes black African ancestry such as persons of African descent or lineage. Black African ancestry may be determined by self reporting as African-Americans, Afro-Americans, Black Americans, being a member of the black race or being a member of the negro race. For example, African Americans or Black Americans are those persons living in North America and having origins in any of the black racial groups of Africa. For example, self-reported persons of black African ancestry may have at least one parent of black African ancestry or at least one grandparent of black African ancestry. Human subjects having a race that includes black African ancestry may also be determined by genetic analysis. Genetic analysis of ancestry may be carried out using unlinked microsatellite markers such as those set out in Smith et al. Am J Hum Genet. 74, 1001-13 (2004).

### Monitoring Effectiveness of Dosing Schedule

Measurement of the level of a leukotriene or inflammatory marker before treatment during and/or after treatment is a method of determining the effectiveness of treatment with the DG-031 dosing schedule of the invention. The efficacy of the dosing schedule is indicated by a decrease in the level of the leukotriene or inflammatory marker, that is, a level of the inflammatory marker during or after treatment that is significantly lower (e.g., significantly lower), than the level of inflammatory marker before treatment (baseline level), is indicative of efficacy. Representative inflammatory markers include: a leukotriene (e.g., LTB4, LTA4), a leukotriene metabolite, C-reactive protein (CRP), serum amyloid A, fibrinogen, interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, matrix metalloprotease type-9, myeloperoxidase (MPO), and N-tyro sine. In a preferred embodiment, the marker is CRP or MPO

One of the preferred inflammatory markers to monitor is serum C-reactive protein (CRP). Generally CRP is measured in serum samples using commercially available enzyme-linked immunosorbent assays (EIA). Consistent across multiple published studies is the finding of a correlation between increased risk for coronary artery disease with increased serum CRP. For example, in the Women's Health Study, CRP was measured in 27,939 apparently healthy American women. The cut-off points for quintiles of serum CRP in women were: less than or equal to 0.49, more than 0.49 to 1.08, more than 1.08 to 2.09, more than 2.09 to 4.19, and more than 4.19 mg CRP per liter, see Ridker, P. M. et al., New England. J. Med., 347: 1557-1565 (2001). In comparison to the lowest quintile, and even when adjusting for age, every quintile more than 0.49 mg CRP per liter was associated with increased risk for coronary heart disease with the highest relative risk of 4.5 seen for those women in the highest quintile of serum CRP (more than 4.19 mg CRP per liter). A similar correlation between increased serum CRP and increased risk for coronary heart disease in women has been reported (Ridker, P. M et al., New Eng. J. Med., 342:836-843 (2000) and Bermudez, E. A. et. al., Arterioscler. Thromb. Vasc. Biol., 22: 1668-1673 (2002)). Men also show a correlation between increased serum inflammatory markers such as CRP and increased risk for coronary heart disease as previously reported (Doggen, C. J. M. et al., J. Internal Med., 248:406-414 (2000) and Ridker, P. M. et al., New England. J. Med., 336: 973-979 (1997)). Quintiles for serum CRP as reported by Doggen et al., were less than 0.65, more than 0.65 to 1.18, more than 1.18 to 2.07, more than 2.07 to 4.23, and more than 4.23 mg CRP per liter. Unlike women, elevated serum CRP correlates with increased relative risk for coronary heart disease only in the 4th and 5th quintiles of CRP (relative risk of 1.7× and 1.9×, respectively). Elevated CRP or other serum inflammatory markers is also prognostic for increased risk of a second myocardial infarct in patients with a previous myocardial infarct (Retterstol, L. et al., Atheroscler., 160: 433-440 (2002)).

Another preferred method of monitoring the effectiveness of the treatment according to a dosing schedule of the invention is by assessing a level of a leukotriene metabolite (e.g., LTB4, LTA4) in the individual (e.g., in a sample of blood, serum, plasma or urine). The invention also encompasses assessing the level of leukotriene metabolite by stimulating production of a leukotriene or a leukotriene metabolite in a test sample from the individual (e.g., a sample comprising neutrophils), using a calcium ionophore, and comparing the level of the leukotriene or leukotriene metabolite with a control level such as a level of the leukotriene or leukotriene metabolite assessed during or after treatment. A level that is significantly lower during or after treatment, than before treatment, is indicative of efficacy of the treatment according to the dosing schedule. Similarly, the invention encompasses methods of assessing response to treatment, by assessing a level of an inflammatory marker in the individual before treatment, and during or after treatment. A level of the inflammatory marker during or after treatment, that is significantly lower than the level of inflammatory marker before treatment, is indicative of efficacy of the treatment.

Because the level of inflammatory markers can be elevated in individuals who are in the target populations of the invention, an assessment of the level of inflammatory markers of the individual both before, and during, treatment according to the DG-031 dosing schedule of the invention will indicate whether the treatment has successfully decreased production of leukotrienes in the arterial vessel wall or in bone-marrow derived inflammatory cells. For example, in one embodiment of the invention, an individual who is a member of a target population as described above (e.g., an individual at risk for MI, ACS, stroke or PAOD, such as an individual who is at-risk due to a FLAP haplotype) can be assessed for response to treatment with a leukotriene synthesis inhibitor, by examining leukotriene levels or leukotriene metabolite levels in the individual. Blood, serum, plasma or urinary leukotrienes (e.g., leukotriene B4 or E4), or ex vivo production of leukotrienes (e.g., in blood samples stimulated with a calcium ionophore to produce leukotrienes), or leukotriene metabolites, can be measured before, and during or after treatment according the dosing schedule. The leukotriene or leukotriene metabolite level before treatment is compared with the leukotriene or leukotriene metabolite level during or after treatment. The efficacy of treatment is indicated by a decrease in leukotriene production: a level of leukotriene or leukotriene metabolite during or after treatment that is significantly lower than the level of leukotriene or leukotriene metabolite before treatment, is indicative of efficacy. A level that is lower during or after treatment can be shown, for example, by decreased serum or urinary leukotrienes, or decreased ex vivo production of leukotrienes, or decreased leukotriene metabolites. A level that is “significantly lower”, as used herein, is a level that is less than the amount that is typically found in control individual(s), or is less in a comparison of disease risk in a population associated with the other bands of measurement (e.g., the mean or median, the highest quartile or the highest quintile) compared to lower bands of measurement (e.g., the mean or median, the other quartiles; the other quintiles).

### Pharmaceutical Compositions

The present invention provides compositions and formulations of the leukotriene synthesis inhibitor, DG-031. For instance, DG-031 can be formulated with one or more physiologically acceptable carriers or excipients to prepare a pharmaceutical composition. The carrier and composition can be sterile injection, inhalation or ocular administration is preferred. The formulation should suit the mode or route of administration and will differ for repeated administration of immediate release dosage forms to achieve a steady state concentration of plasma DG-031 or for controlled or sustained release administration to achieve a steady state concentration of plasma DG-031.

The composition can be a solution, suspension, emulsion, tablet, pill, capsule, sustained release formulation, or powder. The composition can be formulated as a suppository, with traditional additives such as fats, triglycerides or polyethoxlated polymers. Oral formulation can include standard carriers such as pharmaceutical grades of mannitol, lactose, starch, magnesium stearate, polyvinyl pyrollidone, sodium saccharine, cellulose, magnesium carbonate, etc.

A preferred composition of the present invention is a compressed tablet for oral administration that consists essentially of 250 mg of DG-031, 40 mg of corn starch, 96.24 mg of microcrystalline cellulose, 1.24 mg, 10 mg providone 25 [poly(1-vinyl-2-pyrrolidinone 25], 2.52 mg magnesium stearate and purified water having a comprising a film coating consisting essentially of 6 mg methylhydroxypropylcellulose, 1.5 mg polyethylene glycol 4000, 2.5 mg titanium oxide and purified water. The amounts of these ingredients may vary +/−10%. In some variations of the invention, two or more 250 mg DG-031 tablets are administered simultaneously to provide a dose of 500 mg, 750 mg or 1000 mg. High shear wet granulation is a preferred process for manufacturing the tablets where the primary process steps, which would be familiar to one skilled in the art, are blending the powders, followed by high shear wet granulation, wet milling, fluid bed drying, dry milling, tableting and finally pan coating of the tablets.

Suitable pharmaceutically acceptable carriers include but are not limited to water, buffered saline solutions (e.g., NaCl), saline, buffered saline, alcohols, glycerine, ethanol, gum arabic, vegetable oils, benzyl alcohols, polyethylene glycols, gelatin, carbohydrates such as lactose, amylose or starch, dextrose, magnesium stearate, talc, fumed silica, liquid petrolatum, fatty acid esters, hydroxyproplmethyl, polyvinyl pyrolidone, other pharmaceutically acceptable polymers, as well as combinations thereof. The pharmaceutical preparations can, if desired, be mixed with auxiliary agents, e.g., lubricants, cationic crosslinking agents, inert diluents, alkalizing agents, acidifying agents, surfactants, polar solvents, preservatives, stabilizers, wetting agents, emulsifiers, salts for influencing osmotic pressure, buffers, coloring, flavoring and/or aromatic substances and the like which do not deleteriously react with the active agents.

The controlled or sustained release formulation of the invention comprises a formulation of DG-031 and a pH dependent controlled or sustained release matrix. The controlled or sustained release matrix is a solid composition which allows for the prolonged or extended release of active agent at a rate sufficient to maintain therapeutic blood levels of active agent. The controlled or sustained release matrix is designed to provide continuous and prolonged release of DG-031 over a period of from at least 12 to 24 hours. Most preferably the dosage forms of the present invention will provide a release of about 15% after 2 hours, between 20% and 60% after 8 hours and greater than 65% after about 12 hours. Further, the active agent will preferably release 90%-95% or more of the active agent in 12-16 hours.

An example of a delayed release system is a tablet containing DG-031 with an film coating that has a minimum dissolution profile in gastric fluid. Preferred film coatings block the dissolution of a tablet in stomach acid (pH<5.0), but allow for dissolution at a pH greater than 6.0, such coatings include the following: Rhom Rharma's, Eudragit L 100, Eudragit S100, Eudragit L 100-55, Colorcon Surlease, or FMC Aquacoat CPD. Additional exemplary film coatings of the invention comprise methylhydroxypropylcellulose, hypomellose phthalate polymer, ethylcellulose, (not pH triggered) polymethacrylate, hydroxypropyl methylcellulose acetate succinate, cellulose acetate phthalate (CAP) polymer or acrylic resin.

In general, at least one non-pH dependent sustained release agent is present in the composition in an amount of from about 5 wt. % to about 95 wt. %, preferably from about 10 wt. % to about 30 wt. %. It is to be understood, however, that the scope of the present invention is not to be limited to any particular non-pH-dependent sustained release agent. The controlled or sustained release matrix can makeup from about 40% to about 98% of the total weight of a unit dosage form, excluding coatings, according to the present invention. More preferably, the controlled or sustained release matrix will make up from about 5% to about 95% of the total weight of the inventive compositions.

The controlled or sustained release matrix to active agent ratio can be from 20 to 1 to about 1 to 20, and compositions having integer ratios of all possible combinations between these ranges including 1 to 1 are considered embodiments of the present invention.

The controlled or sustained release matrix according to the present invention can include ingredients such as polysaccharides, and other pharmaceutically acceptable excipients. The rate controlling matrix can be any suitable material that forms a matrix which provides sustained release of an alkalized or acidified incorporated active agent, medicament or drug and the like. Pharmaceutically acceptable rate controlling materials which may be used in the present invention include both synthetic and naturally occurring gums and/or polymers and other art-known rate controlling substances. Examples include naturally occurring or modified naturally occurring or synthetic or semi-synthetic polymers and or gums such as, e.g., alginic acid and salts thereof, carrageenan, pectin, xanthan gum, karaya gum, acacia gum, tragacanth gum, locust bean gum, guar gum, beeswax, carnauba wax, modified starch, polyethylene oxide, cetyl alcohol, hydrogenated vegetable oils, and stearyl alcohol, alkylcellulose, hydroxypropylmethylcellulose, methylcellulose, ethylcellulose, and other cellulosic materials or polymers, such as sodium carboxymethylcellulose and hydroxypropyl cellulose, hydroxyethyl cellulose and mixtures of the foregoing. Additional synthetic and/or semisynthetic polymers, which also assist controlled or sustained release, are polymers that resist dilutions in gastric acid conditions and only begin to release their payload in the duodenum, and include, e.g., cellulose acetate phthalate (CAP), vinyl acetate/vinyl chloride copolymers, polyvinyl acetate phthalate (PVAP), hydroxypropyl methylcellulose phthalate, and/or acrylic polymers, such as methacrylic acid ester copolymers, zein, and the like. This list is not meant to be exclusive.

The composition of the present invention may further include other materials such as bulking agents, disintegrating agents, anti-adherants and glidants, lubricants, wetting or emulsifying agents and binding agents. The composition, if desired, can also contain minor amounts pH buffering agents.

Bulking agents include, but are not limited to, microcrystalline cellulose (e.g., Avicel®, FMC Corp., Emcocel®, Mendell Inc.), starches, mannitol, xylitol, dicalcium phosphate (e.g. Emcompress, Mendell Inc.) calcium sulfate (e.g. Compactrol, Mendell Inc.), lactose, sucrose (Dipac, Amstar, and Nutab, Ingredient Technology), dextrose (Emdex, Mendell, Inc.), sorbitol, cellulose powder (Elcema, Degussa, and Solka Floc, Mendell, Inc.) The bulking agent may be present in the composition in an amount of from about 5 wt. % to about 90 wt. %, preferably from about 10 wt. % to about 50 wt. %.

Binding agents which may be employed include, but are not limited to polyvinyl pyrrollidone, starch, methylcellulose, hydroxypropyl methylcellulose, carboxymethyl cellulose, sucrose solution, dextrose solution, acacia, tragacanth and locust bean gum. The binding agent may be present in the composition in an amount of from about 0.2 wt. % to about 10 wt. %, preferably from about 0.5 wt. % to about 5 wt. %.

Disintegrating agents which may be included in the composition include, but are not limited to, microcrystalline cellulose, starches, crospovidone (e.g. Polyplasdone XL, International Specialty Products.), sodium starch glycolate (Explotab, Mendell Inc.), and crosscarmellose sodium (e.g., Ac-Di-Sol, FMC Corp.). The disintegrating agent may be present in the composition in an amount of from about 0.5 wt. % to about 30 wt %, preferably from about 1 wt. % to about 15 wt. %.

Antiadherants and glidants which may be employed in the composition include, but are not limited to, talc, corn starch, silicon dioxide, sodium lauryl sulfate, and metallic stearates. The antiadherant or glidant may be present in the composition in an amount of from about 0.2 wt. % to about 15 wt. %, preferably from about 0.5 wt. % to about 5 wt. %.

Lubricants which may be employed in the composition include, but are not limited to, magnesium stearate, calcium stearate, sodium stearate, stearic acid, sodium stearyl fumarate, hydrogenated cotton seed oil (Sterotex), talc, and waxes, including but not limited to, beeswax, carnuba wax, cetyl alcohol, glyceryl stearate, glyceryl palmitate, glyceryl behenate, hydrogenated vegetable oils, and stearyl alcohol. The lubricant may be present in an amount of from about 0.2 wt. % to about 20 wt. %, preferably from about 0.5 wt. % to about 5 wt. %.

### Modes of Administration

Methods and routes of administering DG-031, either as a repeated dose or as a controlled or sustained release formulation, include but are not limited to, intradermal, pulmonary/inhalants, transdermal, transmucosal, intramuscular, intraperitoneal, intraocular, intravenous, subcutaneous, topical, oral, anal, vaginal, inhalation and intranasal. For repeated dosing, the preferred method of delivery is oral administration of a solid tablet, gel liquid or capsule.

Particular modes of administration contemplated for controlled or sustained release formulations of the invention include solid matrix tablets for oral administration as exemplified herein. The solid controlled or sustained release matrix allows for prolonged retention in the stomach or release of the therapeutic agent in the lower gut. The controlled or sustained release matrix may also be pH dependent, thereby reducing dissolution rate in the stomach or lower gut. The controlled or sustained release matrix can also delay passage through the stomach by expanding in the presence of gastric juices or stomach acid, thereby increasing the time in the stomach as described in U.S. Pat. Nos. 6,290,989, 6,340,475 and 6,776,999. Similarly, the controlled or sustained matrix can transform the shape of the tablet or capsule to prevent passage out of the stomach as described in U.S. Pat. No. 6,488,962. The controlled or sustained release matrix can adhere to the stomach or duodenum mucosa, thereby delaying passage through the stomach or lower gut as described in U.S. Pat. Nos. 6,387,408, 6,428,813 and 6,582,720.

The invention also includes oral administration of multiparticulate capsules and osmotic tablets or other osmotic delivery systems (See U.S. Pat. No. 6,110,498). In addition, the invention includes subcutaneous or parenteral administration by continuous infusion of DG-031 using pumps, infusions and implants. The invention includes transdermal administration such as applying a transdermal patch.

In some variation of the invention, the leukotriene synthesis inhibitor compositions are administered as part of a combinatorial therapy with other agents. For example, most cardiovascular patients, and all which participated in the clinical study described in Example 2, were administered statins (HMG reductase inhibitors). As treatment with DG-031 was effective to reduce LTB4, CRP and MPO levels in patients concurrently being administered statins, the invention includes co-administering DG-031 and a statin for a more effective therapeutic result as described in International Application No. PCT/US2005/003312, filed Jan. 31, 2005, incorporated by reference herein in its entirety. Likewise, the invention includes compositions that comprise a controlled or sustained release formulation of a leukotriene inhibitor in combination with a statin.

The composition can be formulated in accordance with the routine procedures known to those of skill in the art as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette (vial) with a label which indicates the quantity of active agent. Where the composition is to be administered by infusion, it can be dispensed with an infusion bottle containing sterile pharmaceutical grade water, saline or dextrose/water. Where the composition is administered by injection, and the drug is present as a lyophilized solid, an ampule of sterile water for injection or saline can be provided so that the ingredients may be mixed prior to administration.

For topical application, non-sprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material or diluent, is packaged in a squeeze bottle or in admixture with a pressurized volatile, normally gaseous propellant.

Agents described herein can be formulated as neutral or salt forms, or esters or other chemical derivatives that act as prodrugs in vivo, metabolized into the active agent.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. The optimal dose will also depend on the fraction of the drug delivered to the systemic circulation after delivery via a given administration route, as well as drug distribution, metabolism and excretion. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages at recommended dosing intervals.

### Composition Manufacture

The compositions of the present invention may be made by a direct compression method, or by a wet granulation method or other methods known in the art such as roller compaction.

In the direct compression method, the at least one pharmaceutically active agent and other ingredients are sieved through a stainless steel screen to remove lumps and achieve some consistency in particle size. The sieved materials then are charged to a suitable blender, and blended. The blend then is compressed into tablets on a rotary press using appropriate tooling. The compressed tablets may be coated, such as for physical appearance, environmental protection, or programmed/controlled release.

In the wet granulation method, at least one pharmaceutically active agent and other ingredients are granulated with a granulating fluid (e.g., isopropyl alcohol, ethyl alcohol, and/or water) in a planetary mixer, high shear mixer, or fluidized bed granulator. Binding agents may be contained in the granulating fluid, or may be in the dry mix. The wet granules are dried in an oven or fluidized bed dryer, and then sieved through a suitable screen to obtain free flowing granules. The resulting granules were blended with a suitable lubricant and glidant, and the lubricated granules are compressed into tablets on a rotary press using appropriate tooling. If desired, a coating can be applied on the compressed tablets such as for controlled or sustained release.

### EXAMPLES

### Example 1

### Increased LTB4 Production in Activated Neutrophils from MI Patients

A principal bioactive product of one of the two branches of the 5-LO pathway is LTB4. To determine whether the patients with history of MI have increased activity of the 5-LO pathway compared to controls, the LTB4 production in isolated blood neutrophils was measured before and after stimulation in vitro with the calcium ionophore, ionomycin. No difference was detected between the LTB4 production in resting neutrophils from MI patients or controls (results not shown). In contrast, the LTB4 generation by neutrophils from MI patients stimulated with the ionophore was significantly greater than by neutrophils from controls at 15 and 30 minutes, respectively. Moreover, the observed increase in the LTB4 release was largely accounted for by male carriers of haplotype A4, whose cells produced significantly more LTB4 than cells from controls (P value=0.0042) (Table 20). As shown in Table 20, there was also a heightened LTB4 response in males who do not carry HapA but of borderline significance. This could be explained by additional variants in the FLAP gene that have not been uncovered, or alternatively in other genes belonging to the 5-LO pathway, that may account for upregulation in the LTB4 response in some of the patients without the FLAP at-risk haplotype. As shown in Table 20, differences in LTB4 response were not detected in females. However, due to a small sample size this cannot be considered conclusive. Taken together, the elevated levels of LTB4 production of stimulated neutrophils from male carriers of the at-risk haplotype suggest that the disease associated variants in the FLAP gene increase FLAP's response to factors that stimulate inflammatory cells, resulting in increased leukotriene production and increased risk for MI.

Isolation and Activation of Peripheral Blood Neutrophils 50 ml of blood were drawn into EDTA containing vacutainers from 43 MI patients and 35 age and sex matched controls. All blood was drawn at the same time in the early morning after 12 hours of fasting. The neutrophils were isolated using Ficoll-Paque PLUS (Amersham Biosciences).

Briefly, the cell pellets from the Ficoll gradient were harvested and the red blood cells were subsequently lysed in 0.165 M NH4CL for 10 minutes on ice. After washing with PBS, neutrophils were counted and plated at 2×106 cells/ml in 4 ml cultures of 15% Fetal calf serum (FCS) (GIBCO BRL) in RPMI-1640 (GIBCO BRL). The cells were then stimulated with maximum effective concentration of ionomycin (1 μM). At 0, 15, 30, 60 minutes post ionomycin addition 600 μl of culture medium was aspirated and stored at −80° C. for the measurement of LTB4 release as described below. The cells were maintained at 37° C. in a humidified atmosphere of 5% CO2/95% air. All samples were treated with indomethasine (1 μM) to block the cyclooxygenase enzyme. The experimental conditions are described above.

### Ionomycin-Induced Release of LTB4 in Neutrophils

LTB4 Immunoassay Assay Design was used to quantitate LTB4 concentration in supernatant from cultured ionomycin stimulated neutrophils. The assay used is based on the competitive binding technique in which LTB4 present in the testing samples (200 μl) competes with a fixed amount of alkaline phosphatase-labelled LTB4 for sites on a rabbit polyclonal antibody. During the incubation, the polyclonal Ab becomes bound to a goat anti-rabbit Ab coated onto the microplates. Following a wash to remove excess conjugate and unbound sample, a substrate solution is added to the wells to determine the bound enzyme activity. The color development is stopped and the absorbance is read at 405 nm. The intensity of the color is inversely proportional to the concentration of LTB4 in the sample. Each LTB4 measurement using the LTB4 Immunoassay, was done in duplicate.

TABLE 1
LTB4 levels after ionomycin stimulation of isolated neutrophils^{a}

| | After 15 Minutes | | After 30 Minutes | |
|---|---|---|---|---|
| Phenotype (n) | Mean (SD) | P value | Mean (SD) | P value |
| Controls (35) | 4.53 (1.00) | | 4.67 (0.88) | |
| Males (18) | 4.61 (1.10) | | 4.68 (1.07) | |
| Females (17) | 4.51 (0.88) | | 4.67 (0.62) | |
| MI (41) | 5.18 (1.09) | 0.011 | 5.24 (1.06) | 0.016 |
| Carriers (16) | 5.26 (1.09) | 0.027 | 5.27 (1.09) | 0.051 |
| Non-carriers (24) | 5.12 (1.08) | 0.040 | 5.22 (1.03) | 0.035 |
| MI males (28) | 5.37 (1.10) | 0.0033 | 5.38 (1.09) | 0.0076 |
| Carriers (10) | 5.66 (1.04) | 0.0042 | 5.58 (1.12) | 0.013 |
| Non-carriers (18) | 5.20 (1.09) | 0.039 | 5.26 (1.05) | 0.041 |
| MI females (13) | 4.78 (0.95) | 0.46 | 4.95 (0.92) | 0.36 |
| Carriers (6) | 4.59 (0.80) | 0.90 | 4.75 (0.82) | 0.85 |
| Non-carriers (7) | 4.94 (1.04) | 0.34 | 5.12 (0.96) | 0.25 |

^{a}Mean ± SD of log-transformed values of LTB4 levels of ionomycin-stimulated neutrophils from MI patients and controls. Results are shown for two time points: 15 and 30 minutes. The results for males and females and for MI male and female carriers and non-carriers of the at-risk haplotype HapA are shown separately. Two-sided p values corresponding to a standard two-sample test of the difference in the mean values between the MI patients, their various sub-cohorts and the controls are shown.

### Example 2

### Composition of DG-031 Tablets

In the clinical studies described in Examples 3 and 5, the subjects were orally administered DG-031 as a film-coated tablet containing 250 mg of active drug substance. The 250 mg tablet was a round tablet, 410 mg weight. The tablets were stored at 15-30° C. Table 2 lists the components of the DG-031 composition (250 mg Tablets) used in the clinical studies.

TABLE 2
Drug Product Composition (250 mg Tablets)
Each tablet contains:

| | | |
|---|---|---|
| DG-031 | 250 | mg |
| Corn starch | 40 | mg |
| Microcrystalline cellulose | 96.24 | mg |
| Sodium lauryl sulfate | 1.24 | mg |
| Povidone 25 [Poly(1-vinyl-2-pyrrolidinone) 25] | 10 | mg |
| Magnesium stearate | 2.52 | mg |
| Purified Water | | |

Film-coating:

| | | |
|---|---|---|
| Methylhydroxypropylcellulose | 6 | mg |
| Polyethylene glycol 4000 | 1.5 | mg |
| Titanium dioxide | 2.5 | mg |
| Purified Water | | |
| Total weight | 410 | mg |

### Example 3

### Randomized Placebo-Controlled Cross-Over Clinical Trial of DG-031 in Myocardial Infarction Patients (Study No. DG-031-201)

### Patient Population

All patients in the study had a history of MI and were carriers of specific MI-associated haplotypes in the FLAP and/or the LTA₄ hydrolase genes (See U.S. patent application Ser. No. 10/944,272 and PCT Application No. PCT/2004/030582, incorporated by reference in its entirety. The recruitment process included individuals who had previously participated in a population-based study of the genetics of MI (Helgadottir et al. Nat. Genet., 36(3):233-9 2004) Selection of subjects for the study was based on previous haplotype/genotype analysis. Eighty seven percent of the patients enrolled carried some at-risk variant of FLAP, either A3 or AF, 3 single nucleotide polymorphism (SNP) and 2 SNP versions of the at-risk haplotype HapA previously described (Helgadottir et al., Nat. Genet. 2004). Apart from FLAP, allele A of the SNP SG12S25 in the LTA4 hydrolase gene, another gene in the LT pathway, is also associated with MI, as described in PCT application no. PCT/US03/32556 and PCT/US04/30582 incorporated by reference. In particular, among the 13% of the subjects who do not carry an at-risk haplotype of FLAP, all but one carried the at-risk allele A. Four SNP markers were genotyped to define the at-risk variants of the study participants and these markers are set out as Table 3.

TABLE 3
Genotypes used to derive at-risk variants of FLAP and LTA₄ Hydrolase

| Haplotype | Allele | SNP | Allele | SNP | Allele | SNP |
|---|---|---|---|---|---|---|
| A3 (FLAP gene) | G | SG13S25 | T | SG13S114 | A | SG13S32 |
| AF (FLAP gene) | G | SG13S25 | T | SG13S114 | | |
| K (LTA₄-OH gene) | A | SG12S25 | | | | |

The haplotypes carried by each individual were estimated using the program NEMO (version 1.01) and 902 in-house population controls, as previously described in Getarsdottir et al., Nat. Genet. 35: 131-138, 2003. Of over 900 patients identified as eligible by clinical and genotypic criteria, 640 returned their signed consent providing permission to use their genetic and medical data. The genotypes for the FLAP and LTA4 hydrolase genes were subsequently reconfirmed and carriers of at-risk variants in the FLAP and/or LTA4 hydrolase genes were judged eligible for the study if they also met the other inclusion criteria and none of the exclusion criteria (Table 4). The protocol was approved by the National Bioethics Committee of Iceland and all patients who participated gave their informed consent.

TABLE 4
Study Eligibility Criteria

Inclusion criteria
Age 40 to 75.
Carrier of FLAP and/or the LTA₄ hydrolase variants
Documented CAD with previous history of MI
Women of childbearing potential must have a negative urine pregnancy
test at visit 1 and are required to use 2 adequate barrier methods
of contraception throughout the study.
Understanding of the study procedures and agreement to participate in
the study by giving written informed consent.
Exclusion criteria
Confirmed diagnosis of congestive heart failure (CHF).
Any experimental treatment within 2 months of screening or planned
for the following 3 months.
Acute CV event (such as ACS, MI or stroke) within 1 month prior to
enrolment.
Elevated CPK above 3 fold upper normal limit (UNL). Other liver
function tests and kidney function tests above 1.5 fold upper
normal limit.
Immunocompromised subjects, including subjects known to be HIV
positive or with malignant disease and/or on chronic
immunosuppressive therapy.
Subjects known to have positive serology results for HBsAg, HCV Ab.
Treatment with immunosuppressive cytotoxic drugs or corticosteroids
within 6 weeks or during conduct of study.
Major surgery within 6 weeks prior to enrolment.
Any other major intercurrent illness and other condition, which, in the
investigator's judgement, will interfere with the subject's
participation in this study.
Subjects not willing to return for follow-up or with known history of non-
compliance.
Patients who consume more than 2 alcoholic drinks/day or ≧10
drinks/week, or history of alcohol abuse within the past 2 years.
Patients must agree to comply with the restrictions on alcohol
(≦2 drinks/day and <10 drinks/week and no alcohol intake within
48 hours of study visits).
Pregnant or lactating women.
Poor mental function or any other reason to expect patient difficulty in
complying with the requirements of the study.

### Study Conduct

The first patient was enrolled Apr. 5^{th}, 2004 and the study follow-up phase was completed Sep. 14^{th}, 2004. All study participants lived in the Reykjavik metropolitan area or its neighboring townships. All participants were followed by the designated cardiologists at the University Hospital of Iceland, at their outpatient or private clinics and all subjects had participated in a study on the genetics of MI as described in Helgadottir et al. Nat Genet., 36(3):233-9 2004. A medical history was completed, including detailed information on co-morbidities, concomitant medications and specific details about the subject's cardiovascular history, including current status. All participants were fasting and had not taken their medications prior to the study visit. Cardiologists examined the patients at all 8 visits and completed the case report forms. All blood was collected and bus (San Francisco, Calif.). All blood specimens used for the biomarker studies were processed within 2 hours of blood sampling. The trial was double-blinded and treating physicians were blinded to randomization to drug regimen, including dosing and placebo.

### Treatment Groups

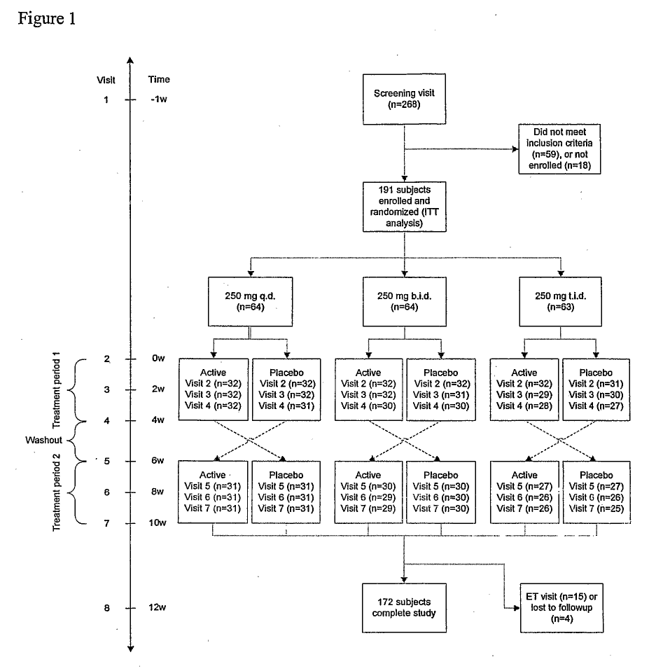

Patients who met the study eligibility criteria were enrolled and randomized into 3 different dose-level groups: (1) 64 patients on 250 mg/day therapy with DG-031 (250 mg q.d.), vs placebo; (2) 64 patients on 500 mg/day therapy with DG-031 (250 mg b.i.d.) vs placebo; and (3) 63 patients on 750 mg/day therapy with DG-031 (250 mg t.i.d.) vs placebo. The 750 mg/day dose was well tolerated in previous phase I-II human studies in asthma (Dahlen et al. Thorax 523: 348-354, 1997; Hamilton et al., Thorax 52: 348-54, 1997). All patients received 3 tablets per day. Treatment periods, 4 weeks in duration each, were separated by a 2-week washout period. The placebo tablets were identical in shape, color, form and taste to the active tablets except that they contained no active drug ingredients. Treatment with DG-031 or placebo was in addition to the subject's standard care, including all medications and treatment plan as prescribed by the subject's cardiologist prior to enrolment. The cross-over study design is summarized in FIG. 1. A total of 191 subjects were enrolled, with 172 completing all 8 visits or 8 patients short of the target of 180 patients.

### Endpoints

The primary objective was to determine whether the FLAP inhibitor has a statistically significant effect, compared to placebo, on one or more biomarkers of MI risk, including: (1) ionomycin-induced LTB₄ and MPO release by neutrophils ex vivo; (2) MPO, CRP, N-tyrosine, Lp-PLA₂ or amyloid A in serum, or (3) LTE₄ in urine. The secondary objective was to determine whether the effect of DG-031 was dose-dependent. The tertiary objective was to assess other biomarkers (See Results). Evaluation of safety and tolerability of the drug was also a primary endpoint.

### Data Analysis, Randomization and Statistical Considerations

All data were analyzed according to a pre-established statistical analysis plan (SAP) and by intention-to-treat (ITT). Each arm of the study, as well as pooled sets (combining dose levels), was considered for the primary analysis. Each such set is an AB/BA cross-over design and in the primary analysis of efficacy, the levels of biomarkers of MI risk at the end of the treatment periods (visits 4 and 7) were used as primary response variables. The difference between DG-031 and placebo treatment was the primary outcome, assessed separately for each of the biomarkers. Treatment effect was tested using a two-sample t-test on the period differences for suitably transformed response variables, under an assumption of normality of the transformed data. We report treatment effect as one half of the observed mean differences in the two-sample t-test, with a 95% CI. No pre-tests for carry-over effect were performed as a part of the primary analysis. Tests for carry-over were done and are reported separately from results of primary analysis. All hypotheses were tested at a two-sided nominal significance level of 0.05 and p-values based on t-tests are reported. In addition, for the primary efficacy endpoints where the effects of the two highest doses on 10 primary variables were studied, a randomization test was performed which corrects for multiple testing and ensures that the key results are not affected by distributional concerns. In particular, 1 million permutations of the patients into the different study tracks were performed, generating a reference distribution for the maximum of the 10 t-statistics of the biomarkers under the null hypothesis of no drug effect. By comparing the observed t-statistic of each of the 10 biomarkers to this reference distribution, empirical p-values were computed. As was stated in the SAP prior to unblinding, it was considered likely that experimental manipulations would alter the effect of the drug on two of the primary markers; this was indeed the case and we are only reporting directly on 8 of the 10 primary markers but all 10 are included in the randomization test.

To cancel out potential seasonal effects, carry-over effects were also studied with two-sample t-tests that compare measurements of the AB (drug/placebo) group with the measurements of the BA (placebo/drug) group. To estimate the effect of the drug at visit 3 for the AB group, (v3−v2), with v3 and v2 denoting, respectively, measurements at visit 3 and visit 2, was used. Similarly (v4−v2) and (v5−v2) were used to estimate effects at visits 4 and 5. For estimating the effect at visit 6, [(v6−v2)+(v3−v2)] was used. Note that v6 from the BA group includes the drug effect after two weeks which cancels out the drug effect at visit 3 from the AB group. Similarly, [(v7−v2)+(v4−v2)] was used to estimate the effect at visit 7. The two higher dose AB groups were used for all visits. All 3 BA groups were used for visits 3, 4 and 5 since they had all received the same treatment until visit 5, but only the two higher dose BA groups are used for visits 6 and 7.

The sample size for this study was chosen so that each of the three arms provided, after up to 5% dropout, at least 80% power (with α=0.05, two-sided) to detect a relative lowering of 15% for a log-normal response variable, given that an assay for that variable has a coefficient of variation of 20% and the intra-person coefficient of variation is as high as 25%. Based on these assumptions, the recruitment target included 180 subjects with randomization into 3 different dose-level groups as described above.

A study flowchart is shown in FIG. 1. At the enrolment visit an independent study nurse who was blinded to the drug content, dispensed medication kits according to a computer generated randomization list. Randomization of study patients was stratified according to sex. For both strata a permuted block design with block size 12 was used to assign patients into each of the six sequences of the study. All biomarkers were transformed using a shifted log transform (transformed value is natural log of original value plus a shifting constant for each assay). Missing data were filled in using a simple last observation carried forward (LOCF) scheme, in cases where no previous measurement existed, next observation was carried back. Statistical outliers for data sets were brought in based on IQR distance from median, a process that was decided before unblinding data, and implemented blind to the treatment tracks.

### Genotyping and Biomarker Assays

DNA genotyping of FLAP and LTA₄ hydrolase variants SNPs genotyping within the FLAP and LTA4 hydrolase genes was performed using SNP-based Taqman platform (ABI) as previously described in Helgadottir et al. Nat. Genet., 36(3):233-9 2004 (Table 2 above).

Biomarker measurements: The ELISA and mass spectrometry assays used are described in the following references: Profita et al., J Pharmacol Exp Ther. 300:868-75, 2002; Roberts et al., Clin Chem. 47:418-25, 2001; Dada et al., Expert Rev Mol Diagn. 2:17-22, 2002; Tsen et al., Clin Chem. 49:810-3, 2003; Sigurdardottir et al., J Intern Med. 252:440-7, 2002; Blum et al., Am J Cardiol. 86:892-895, 2000; Malik et al., Lancet. 358:971-6, 2001; Blum et al., J Am Coll Cardiol. 35:271-6, 2000; Andersen et al., Arthritis Rheum. 43:1085-93, 2000; Yang et al., Am J Gastroenterol. 97:126-32, 2002, Link et al., J Immunol. 164:436-42, 2000; Mohamed-Ali et al., J Clin Endocrinol Metab. 86:5864-9, 2001; Ryan et al., J Infect Dis. 184:699-706, 2001; van der Vliet et al., Respir Res. 1:67-72, 2000; Goulet et al., J. Immunol. 164:4899-907, 2000; McDonald et al., J Immunol Methods. 144:149-55, 1991; Klein et al., J Immunol. 167:524-31, 2001; Gibson et al., Chest. 119:1329-36, 2001. Apart from measurements in plasma, LTB₄ and MPO were also measured in whole blood preparations ex vivo following ionomycin-activation of leukocytes, using ELISA and mass spectrometry. Both dose and time dependent stimulations were performed to determine the maximum LTB₄ and MPO output of the cells. Correction was made for white blood cell count, as the amount of these mediators produced is proportional to the number of cells in a fixed volume. On the log scale the adjustment was based on a linear model, with coefficients determined empirically at time of blind review. Several tertiary markers were also measured including: IL-6, IL-12p40, TNF-α, MMP-9, sICAM, sVCAM, P-selectin, E-selectin, MCP-1 and oxidised LDL.

### Baseline Characteristics of the Patients

The study subjects were randomly assigned to six treatment sequences (FIG. 1); two sequences (active-placebo and placebo-active) for each of the three dose regiments (250 mg, 500 mg and 750 mg). All 191 subjects were analysed as randomized in the intention-to-treat analysis using the LOCF scheme described above. As shown in Table 5 there are no differences in the baseline characteristics of the study subjects between the study sequences. Table 6 shows some summary statistics for baseline values of the biomarker data.

TABLE 5
Baseline characteristics of the study cohort

| | 250 mg/day | | | | 500 mg/day | | | | 750 mg/day | | | |
|---|---|---|---|---|---|---|---|---|---|---|---|---|
| | Active- | | Placebo- | | Active- | | Placebo- | | Active- | | Placebo- | |
| | placebo | | active | | placebo | | active | | placebo | | active | |
| Characteristic | (n = 32) | | (n = 32) | | (n = 32) | | (n = 32) | | (n = 32) | | (n = 31) | |
| Demography | | | | | | | | | | | | |
| Male/Female | 24/8 | | 24/8 | | 24/8 | | 24/8 | | 24/8 | | 24/7 | |
| Age (SD), years | 66 | (8) | 66 | (8) | 65 | (7) | 67 | (7) | 64 | (8) | 67 | (7) |
| Age range, years | 47-75 | | 47-75 | | 51-75 | | 52-75 | | 47-75 | | 56-75 | |
| Age >60 years, % | 78% | | 75% | | 78% | | 78% | | 69% | | 74% | |
| Weight (SD), kg | 86 | (11) | 87 | (12) | 86 | (14) | 92 | (18) | 91 | (13) | 93 | (19) |
| Height (SD), cm | 173 | (8) | 174 | (7) | 173 | (8) | 174 | (9) | 174 | (7) | 173 | (10) |
| BMI (SD), kg/m² | 29 | (3) | 29 | (3) | 29 | (4) | 30 | (6) | 30 | (4) | 31 | (5) |
| Cardiovascular history | | | | | | | | | | | | |
| Two or more prev. infarcts | 3 | (9%) | 6 | (19%) | 3 | (9%) | 7 | (22%) | 6 | (19%) | 8 | (26%) |
| Time since last MI (mo's) | 146 | (63) | 137 | (73) | 143 | (65) | 121 | (68) | 129 | (71) | 131 | (59) |
| Hypertension (current) | 5 | (16%) | 10 | (31%) | 4 | (12%) | 4 | (12%) | 8 | (25%) | 7 | (23%) |
| Diabetes | 10 | (31%) | 8 | (25%) | 6 | (19%) | 12 | (38%) | 8 | (25%) | 10 | (32%) |
| Haplotype frequency | | | | | | | | | | | | |
| A3 carrier (FLAP) | 8 | (25%) | 7 | (22%) | 8 | (25%) | 5 | (16%) | 11 | (34%) | 13 | (42%) |
| AF carrier (FLAP)* | 29 | (91%) | 27 | (84%) | 28 | (88%) | 28 | (88%) | 28 | (88%) | 26 | (84%) |
| SG12S25 carrier (LTA₄-OH) | 31 | (97%) | 28 | (88%) | 30 | (90%) | 31 | (97%) | 25 | (78%) | 28 | (90%) |
| Relevant medication | | | | | | | | | | | | |
| Statins (%) | 27 | (84%) | 28 | (88%) | 26 | (81%) | 28 | (88%) | 25 | (78%) | 27 | (87%) |
| Other chol'l low'ng drug (%) | 0 | (0%) | 0 | (0%) | 3 | (9%) | 1 | (3%) | 1 | (3%) | 1 | (3%) |
| Aspirin (%) | 28 | (88%) | 28 | (88%) | 28 | (88%) | 25 | (78%) | 27 | (84%) | 26 | (84%) |
| Nitrates (%) | 13 | (41%) | 12 | (38%) | 10 | (31%) | 8 | (25%) | 8 | (25%) | 12 | (39%) |
| Ca-channel blockers (%) | 9 | (28%) | 6 | (19%) | 9 | (28%) | 7 | (22%) | 7 | (22%) | 8 | (26%) |
| ACE-inhibitors (%) | 7 | (22%) | 10 | (31%) | 12 | (38%) | 10 | (31%) | 10 | (31%) | 13 | (42%) |
| Beta-blockers (%) | 22 | (69%) | 23 | (72%) | 23 | (72%) | 18 | (56%) | 24 | (75%) | 22 | (71%) |
| Diuretics (%) | 9 | (28%) | 13 | (41%) | 7 | (22%) | 7 | (22%) | 11 | (34%) | 9 | (29%) |
| Plasma lipids | | | | | | | | | | | | |
| Cholesterol (SD), mmol/L | 5.0 | (1.0) | 5.0 | (0.8) | 5.2 | (1.0) | 4.8 | (1.1) | 5.2 | (1.2) | 5.0 | (1.0) |
| HDL (SD), mmol/L | 1.4 | (0.3) | 1.4 | (0.3) | 1.5 | (0.3) | 1.4 | (0.5) | 1.4 | (0.4) | 1.4 | (0.3) |
| LDL (SD), mmol/L | 3.0 | (1.0) | 3.0 | (0.7) | 3.1 | (1.0) | 2.9 | (1.0) | 3.2 | (1.0) | 3.0 | (0.9) |
| Triglycerides (SD), mmol/L | 1.4 | (0.8) | 1.5 | (0.7) | 1.7 | (1.6) | 1.3 | (0.7) | 1.4 | (0.7) | 1.4 | (0.6) |
| Blood pressure | | | | | | | | | | | | |
| Diastolic (SD), mmHg | 79 | (8) | 78 | (7) | 81 | (11) | 79 | (9) | 78 | (12) | 78 | (7) |
| Systolic (SD), mmHg | 137 | (13) | 133 | (19) | 139 | (22) | 136 | (17) | 141 | (22) | 140 | (17) |
| Smoking habits | | | | | | | | | | | | |
| Never smoked | 9 | (28%) | 4 | (13%) | 5 | (16%) | 3 | (9%) | 8 | (25%) | 7 | (23%) |
| Prior history of smoking | 18 | (56%) | 20 | (63%) | 19 | (59%) | 24 | (75%) | 19 | (59%) | 16 | (52%) |
| Current smoker | 5 | (16%) | 8 | (25%) | 8 | (25%) | 5 | (16%) | 5 | (16%) | 8 | (26%) |
| Alcohol use | | | | | | | | | | | | |
| Never used alcohol | 4 | (13%) | 4 | (13%) | 2 | (6%) | 5 | (16%) | 5 | (16%) | 5 | (16%) |
| Prior use of alcohol | 1 | (3%) | 5 | (16%) | 4 | (13%) | 3 | (9%) | 4 | (13%) | 2 | (6%) |
| Current use of alcohol | 27 | (84%) | 23 | (72%) | 26 | (81%) | 24 | (75%) | 23 | (72%) | 24 | (77%) |
| Demography | | | | | | | | | | | | |
| Male/Female | 24/8 | | 24/8 | | 24/8 | | 24/8 | | 24/8 | | 24/7 | |
| Age (SD), years | 66 | (8) | 66 | (8) | 65 | (7) | 67 | (7) | 64 | (8) | 67 | (7) |
| Age range, years | 47-75 | | 47-75 | | 51-75 | | 52-75 | | 47-75 | | 56-75 | |
| Age >60 years, % | 78% | | 75% | | 78% | | 78% | | 69% | | 74% | |
| Weight (SD), kg | 86 | (11) | 87 | (12) | 86 | (14) | 92 | (18) | 91 | (13) | 93 | (19) |
| Height (SD), cm | 173 | (8) | 174 | (7) | 173 | (8) | 174 | (9) | 174 | (7) | 173 | (10) |
| BMI (SD), kg/m² | 29 | (3) | 29 | (3) | 29 | (4) | 30 | (6) | 30 | (4) | 31 | (5) |
| Cardiovascular history | | | | | | | | | | | | |
| Two or more prev. infarcts | 3 | (9%) | 6 | (19%) | 3 | (9%) | 7 | (22%) | 6 | (19%) | 8 | (26%) |
| Time since last MI (mo's) | 146 | (63) | 137 | (73) | 143 | (65) | 121 | (68) | 129 | (71) | 131 | (59) |
| Hypertension (current) | 5 | (16%) | 10 | (31%) | 4 | (12%) | 4 | (12%) | 8 | (25%) | 7 | (23%) |
| Diabetes | 10 | (31%) | 8 | (25%) | 6 | (19%) | 12 | (38%) | 8 | (25%) | 10 | (32%) |
| Haplotype frequency | | | | | | | | | | | | |
| A3 carrier (FLAP) | 8 | (25%) | 7 | (22%) | 8 | (25%) | 5 | (16%) | 11 | (34%) | 13 | (42%) |
| AF carrier (FLAP)* | 29 | (91%) | 27 | (84%) | 28 | (88%) | 28 | (88%) | 28 | (88%) | 26 | (84%) |
| SG12S25 carrier (LTA₄-OH) | 31 | (97%) | 28 | (88%) | 30 | (90%) | 31 | (97%) | 25 | (78%) | 28 | (90%) |
| Relevant medication | | | | | | | | | | | | |
| Statins (%) | 27 | (84%) | 28 | (88%) | 26 | (81%) | 28 | (88%) | 25 | (78%) | 27 | (87%) |
| Other chol'l low'ng drug (%) | 0 | (0%) | 0 | (0%) | 3 | (9%) | 1 | (3%) | 1 | (3%) | 1 | (3%) |
| Aspirin (%) | 28 | (88%) | 28 | (88%) | 28 | (88%) | 25 | (78%) | 27 | (84%) | 26 | (84%) |
| Nitrates (%) | 13 | (41%) | 12 | (38%) | 10 | (31%) | 8 | (25%) | 8 | (25%) | 12 | (39%) |
| Ca-channel blockers (%) | 9 | (28%) | 6 | (19%) | 9 | (28%) | 7 | (22%) | 7 | (22%) | 8 | (26%) |
| ACE-inhibitors (%) | 7 | (22%) | 10 | (31%) | 12 | (38%) | 10 | (31%) | 10 | (31%) | 13 | (42%) |
| Beta-blockers (%) | 22 | (69%) | 23 | (72%) | 23 | (72%) | 18 | (56%) | 24 | (75%) | 22 | (71%) |
| Diuretics (%) | 9 | (28%) | 13 | (41%) | 7 | (22%) | 7 | (22%) | 11 | (34%) | 9 | (29%) |
| Plasma lipids | | | | | | | | | | | | |
| Cholesterol (SD), mmol/L | 5.0 | (1.0) | 5.0 | (0.8) | 5.2 | (1.0) | 4.8 | (1.1) | 5.2 | (1.2) | 5.0 | (1.0) |
| HDL (SD), mmol/L | 1.4 | (0.3) | 1.4 | (0.3) | 1.5 | (0.3) | 1.4 | (0.5) | 1.4 | (0.4) | 1.4 | (0.3) |
| LDL (SD), mmol/L | 3.0 | (1.0) | 3.0 | (0.7) | 3.1 | (1.0) | 2.9 | (1.0) | 3.2 | (1.0) | 3.0 | (0.9) |
| Triglycerides (SD), mmol/L | 1.4 | (0.8) | 1.5 | (0.7) | 1.7 | (1.6) | 1.3 | (0.7) | 1.4 | (0.7) | 1.4 | (0.6) |
| Blood pressure | | | | | | | | | | | | |
| Diastolic (SD), mmHg | 79 | (8) | 78 | (7) | 81 | (11) | 79 | (9) | 78 | (12) | 78 | (7) |
| Systolic (SD), mmHg | 137 | (13) | 133 | (19) | 139 | (22) | 136 | (17) | 141 | (22) | 140 | (17) |
| Smoking habits | | | | | | | | | | | | |
| Never smoked | 9 | (28%) | 4 | (13%) | 5 | (16%) | 3 | (9%) | 8 | (25%) | 7 | (23%) |
| Prior history of smoking | 18 | (56%) | 20 | (63%) | 19 | (59%) | 24 | (75%) | 19 | (59%) | 16 | (52%) |
| Current smoker | 5 | (16%) | 8 | (25%) | 8 | (25%) | 5 | (16%) | 5 | (16%) | 8 | (26%) |
| Alcohol use | | | | | | | | | | | | |
| Never used alcohol | 4 | (13%) | 4 | (13%) | 2 | (6%) | 5 | (16%) | 5 | (16%) | 5 | (16%) |
| Prior use of alcohol | 1 | (3%) | 5 | (16%) | 4 | (13%) | 3 | (9%) | 4 | (13%) | 2 | (6%) |
| Current use of alcohol | 27 | (84%) | 23 | (72%) | 26 | (81%) | 24 | (75%) | 23 | (72%) | 24 | (77%) |

*a common low-risk haplotype (RR 1.3) carried by 87% of study subjects

TABLE 6

| Assay | Unit | Min | 1. quart. | Median | 3. quart. | Max. | n |
|---|---|---|---|---|---|---|---|
| Primary objectives | | | | | | | |
| Amyloid A | ng/ml | 5614 | 10930 | 13920 | 21250 | 1257000 | 191 |
| Hs-CRP | pg/ml | 0.3 | 1.1 | 1.9 | 4.0 | 65.0 | 191 |
| LTE₄ in urine | pg/ml | 226.3 | 567.8 | 745.1 | 936.1 | 3636.0 | 189 |
| Lp-PLA₂ | μg/ml | 61.49 | 194.60 | 226.90 | 277.10 | 668.60 | 191 |
| MPO in plasma | ng/ml | 12.39 | 26.62 | 35.46 | 52.74 | 167.10 | 190 |
| N-tyrosine | nM | 10.65 | 14.63 | 23.84 | 42.63 | 5030 | 180 |
| LTB₄ in whole blood^{†} | pg/ml | 3736 | 28620 | 47740 | 73850 | 440600 | 189 |
| LTB₄ in w.b.*, corr. for wbc^{†,‡} | | 336.5 | 2114.0 | 3373.0 | 5058.0 | 12450.0 | 189 |
| MPO in whole blood | ng/ml | 237.0 | 521.6 | 702.8 | 940.8 | 2433.0 | 189 |
| MPO in w.b.*, corr. for wbc^{‡} | | 46.01 | 84.88 | 111.40 | 136.80 | 289.80 | 189 |
| White blood cell count^{§} | 10⁹/L | 2.9 | 5.4 | 6.2 | 7.5 | 13.6 | 191 |
| Tertiary objectives | | | | | | | |
| ICAM | ng/ml | 163.7 | 248.4 | 280.7 | 332.8 | 788.3 | 191 |
| IL12p40 | pg/ml | 41.22 | 107.00 | 137.80 | 188.20 | 516.10 | 191 |
| IL6 | pg/ml | 0.3476 | 1.9650 | 2.5000 | 3.4510 | 65.97 | 191 |
| MCP-1 | pg/ml | 200.4 | 311.8 | 362.0 | 438.2 | 2391 | 190 |
| MMP 9 | ng/ml | 75.36 | 376.10 | 490.40 | 706.80 | 1712.0 | 191 |
| Oxidized - LDL | mU/L | 25480 | 54350 | 63720 | 74910 | 176600 | 191 |
| TNF-α | pg/ml | 0.6882 | 1.2410 | 1.6320 | 2.1530 | 10.360 | 166 |
| sE-Selectin | ng/ml | 36.63 | 53.30 | 66.04 | 76.52 | 184.30 | 191 |
| sP-Selectin | ng/ml | 44.43 | 102.80 | 128.40 | 152.90 | 1489.0 | 187 |
| sVCAM | ng/ml | 272.9 | 387.6 | 431.3 | 491.8 | 984.0 | 189 |

*w.b. = whole blood
^{†}baseline is not available for LTB₄ measured using mass spectrometry
^{‡}corr. for wbc = corrected for white blood cell count
^{§}WBC is not part of the primary objectives, but is included here due to the wbc correction used for LTB₄ and MPO.

### Outcome—Primary Efficacy Endpoints

For the primary efficacy endpoint, as specified in the statistical analysis plan (SAP), 10 variables were considered in the pooled set of subjects on 500 mg and 750 mg arms (Table 7 and Table 8). The primary efficacy endpoint of the study was confirmed by showing that DG-031 reduces levels of LTB₄ produced by ionomycin-activated neutrophils ex vivo for the pooled set of 500 mg and 750 mg arms (17% [6%,27%], nominal p=0.004), which is statistically significant after correction for multiple testing using the randomization procedure (corrected p=0.02). On the other hand urine levels of LTE₄ were increased by 21% for the pooled dose set ([13%,30%], p<0.001, corrected p<0.001).

TABLE 7
Treatment effect based on two sample t-test for the treatment groups, the pooled sets for the two
highest doses and all doses (natural log scale)

| | | | | | |
|---|---|---|---|---|---|
| | 250 mg/day | 500 mg/day | 750 mg/day | 500 & 750 mg/day | 250, 500 & 750 |
| Assay | (n = 64) | (n = 64) | (n = 63) | (n = 127) | mg/day (n = 191) |

Primary objectives

| | | | | | |
|---|---|---|---|---|---|
| Amyloid A | 0.03 [−0.09, 0.15] | −0.05 [−0.17, 0.06] | −0.01 [−0.11, 0.09] | −0.03 [−0.11, 0.05] | −0.01 [−0.07, 0.05] |
| | (p = 0.61) | (p = 0.36) | (p = 0.90) | (p = 0.43) | (p = 0.77) |
| Hs-CRP | 0.05 [−0.14, 0.24] | 0.09 [−0.09, 0.26] | 0.04 [−0.13, 0.21] | 0.06 [−0.06, 0.18] | 0.06 [−0.04, 0.16] |
| | (p = 0.59) | (p = 0.34) | (p = 0.66) | (p = 0.32) | (p = 0.26) |
| Lp-PLA₂ | 0.05 [−0.03, 0.12] | 0.03 [−0.04, 0.10] | 0.09 [0.03, 0.15] | 0.06 [0.01, 0.10] | 0.05 [0.01, 0.09] |
| | (p = 0.24) | (p = 0.37) | (p = 0.006) | (p = 0.01) | (p = 0.007) |
| LTB₄ in w.b.*, mass | −0.11 [−0.29, 0.06] | −0.09 [−0.28, 0.11] | −0.26 [−0.46, −0.06] | −0.17 [−0.31, −0.04] | −0.15 [−0.26, −0.05] |
| spec.^{†} | (p = 0.19) | (p = 0.38) | (p = 0.01) | (p = 0.01) | (p = 0.005) |
| LTB₄ in w.b.*, corr. | −0.11 [−0.28, 0.05] | −0.08 [−0.26, 0.09] | −0.30 [−0.49, −0.11] | −0.19 [−0.32, −0.06] | −0.16 [−0.27, −0.06] |
| for wbc^{‡}, m.s.^{§} | (p = 0.18) | (p = 0.35) | (p = 0.003) | (p = 0.004) | (p = 0.002) |
| LTB₄ in whole blood^{†} | −0.13 [−0.35, 0.09] | −0.19 [−0.44, 0.06] | −0.30 [−0.56, −0.04] | −0.24 [−0.42, −0.07] | −0.21 [−0.34, −0.07] |
| | (p = 0.24) | (p = 0.13) | (p = 0.02) | (p = 0.007) | (p = 0.004) |
| LTB₄ in w.b.*, corr. | −0.13 [−0.35, 0.08] | −0.18 [−0.42, 0.05] | −0.34 [−0.59, −0.09] | −0.26 [−0.43, −0.09] | −0.22 [−0.35, −0.08] |
| for wbc^{†,‡} | (p = 0.22) | (p = 0.12) | (p = 0.009) | (p = 0.003) | (p = 0.001) |
| LTE₄ in urine | 0.14 [0.03, 0.24] | 0.15 [0.05, 0.24] | 0.24 [0.14, 0.34] | 0.19 [0.12, 0.26] | 0.17 [0.12, 0.23] |
| | (p = 0.01) | (p = 0.003) | (p < 0.001) | (p < 0.001) | (p < 0.001) |
| MPO in plasma | −0.07 [−0.22, 0.07] | 0.08 [−0.04, 0.21] | −0.04 [−0.17, 0.09] | 0.02 [−0.07, 0.11] | −0.01 [−0.09, 0.06] |
| | (p = 0.32) | (p = 0.20) | (p = 0.49) | (p = 0.68) | (p = 0.76) |
| MPO in whole blood^{†} | 0.01 [−0.08, 0.11] | −0.01 [−0.13, 0.11] | −0.11 [−0.22, 0.00] | −0.06 [−0.14, 0.02] | −0.04 [−0.10, 0.03] |
| | (p = 0.78) | (p = 0.85) | (p = 0.06) | (p = 0.14) | (p = 0.27) |
| MPO in w.b.*, corr. | 0.01 [−0.08, 0.11] | 0.00 [−0.11, 0.12] | −0.13 [−0.24, −0.02] | −0.06 [−0.14, 0.02] | −0.04 [−0.10, 0.02] |
| for wbc^{‡} | (p = 0.76) | (p = 0.94) | (p = 0.02) | (p = 0.12) | (p = 0.24) |
| N-tyrosine | −0.03 [−0.15, 0.09] | −0.03 [−0.13, 0.08] | 0.03 [−0.08, 0.14] | 0.00 [−0.07, 0.08] | −0.01 [−0.07, 0.05] |
| | (p = 0.60) | (p = 0.60) | (p = 0.56) | (p = 0.96) | (p = 0.78) |

Tertiary objectives

| | | | | | |
|---|---|---|---|---|---|
| ICAM | 0.00 [−0.04, 0.03] | 0.00 [−0.04, 0.03] | −0.03 [−0.06, 0.00] | −0.02 [−0.04, 0.00] | −0.01 [−0.03, 0.01] |
| | (p = 0.83) | (p = 0.81) | (p = 0.03) | (p = 0.10) | (p = 0.16) |
| IL12p40 | 0.01 [−0.04, 0.06] | 0.02 [−0.04, 0.08] | 0.01 [−0.04, 0.06] | 0.01 [−0.02, 0.05] | 0.01 [−0.02, 0.04] |
| | (p = 0.69) | (p = 0.53) | (p = 0.70) | (p = 0.46) | (p = 0.40) |
| IL6 | −0.02 [−0.13, 0.09] | 0.06 [−0.03, 0.16] | −0.01 [−0.10, 0.09] | 0.03 [−0.04, 0.09] | 0.01 [−0.05, 0.07] |
| | (p = 0.68) | (p = 0.19) | (p = 0.87) | (p = 0.40) | (p = 0.69) |
| MCP-1 | −0.02 [−0.07, 0.03] | 0.02 [−0.03, 0.08] | −0.03 [−0.08, 0.03] | 0.00 [−0.04, 0.04] | −0.01 [−0.04, 0.02] |
| | (p = 0.51) | (p = 0.35) | (p = 0.32) | (p = 0.98) | (p = 0.69) |
| MMP 9 | −0.03 [−0.12, 0.05] | 0.02 [−0.06, 0.11] | −0.02 [−0.11, 0.06] | 0.00 [−0.06, 0.06] | −0.01 [−0.06, 0.04] |
| | (p = 0.47) | (p = 0.58) | (p = 0.60) | (p = 0.97) | (p = 0.69) |
| Oxidized —LDL | 0.00 [−0.08, 0.07] | 0.02 [−0.07, 0.11] | 0.06 [−0.03, 0.16] | 0.04 [−0.02, 0.11] | 0.03 [−0.02, 0.08] |
| | (p = 0.91) | (p = 0.65) | (p = 0.16) | (p = 0.18) | (p = 0.28) |
| sE-Selectin | 0.03 [−0.03, 0.09] | −0.01 [−0.06, 0.04] | −0.04 [−0.09, 0.01] | −0.02 [−0.06, 0.01] | 0.00 [−0.04, 0.03] |
| | (p = 0.30) | (p = 0.82) | (p = 0.11) | (p = 0.20) | (p = 0.75) |
| sP-Selectin | −0.02 [−0.11, 0.06] | 0.00 [−0.08, 0.08] | 0.09 [0.01, 0.16] | 0.04 [−0.02, 0.10] | 0.02 [−0.03, 0.07] |
| | (p = 0.58) | (p = 0.97) | (p = 0.03) | (p = 0.15) | (p = 0.40) |
| sVCAM | 0.00 [−0.05, 0.04] | −0.01 [−0.06, 0.04] | −0.03 [−0.07, 0.02] | −0.02 [−0.05, 0.01] | −0.01 [−0.04, 0.01] |
| | (p = 0.85) | (p = 0.60) | (p = 0.24) | (p = 0.24) | (p = 0.28) |
| TNF-α | 0.00 [−0.08, 0.09] | −0.02 [−0.10, 0.07] | 0.01 [−0.07, 0.08] | 0.00 [−0.06, 0.06] | 0.00 [−0.05, 0.05] |
| | (p = 0.93) | (p = 0.70) | (p = 0.85) | (p = 0.90) | (p = 0.95) |

*w.b. = whole blood
^{†}measurement is not part of the primary analysis wrt adjustment for multiple testing
^{‡}corr. for wbc = corrected for white blood cell count
^{§}m.s. = mass spec. = mass spectrometry

TABLE 8
Exact and corrected p-values based on randomization

| | Nominal | Randomized | Corrected |
|---|---|---|---|
| Assay | p-value | p-value | p-value |
| Amyloid A | 0.43 | 0.43 | 0.99 |
| Hs-CRP | 0.32 | 0.33 | 0.96 |
| Lp-PLA₂ | 0.01 | 0.005 | 0.08 |
| LTB₄ in whole blood, | 0.004 | 0.003 | 0.02 |
| corr. for wbc^{‡}, m.s.^{§} | | | |
| LTE₄ in urine | <0.001 | <0.001 | <0.001 |
| MPO in plasma | 0.68 | 0.67 | 1.00 |
| MPO in whole blood, corr. | 0.11 | 0.07 | 0.60 |
| for wbc^{‡} | | | |
| N-tyrosine | 0.96 | 0.97 | 1.00 |

^{‡}corr. for wbc = corrected for white blood cell count
^{§}m.s. = mass spec. = mass spectrometry

### Outcome—Variables of Primary Objectives

As shown in Table 7, the maximum reduction in LTB₄ and MPO production amounted to 26% for LTB₄ ([10%,39%], p=0.003) and 12% for MPO ([2%,21%], p=0.02) at the 750 mg/day dose of DG-031. DG-031 also reduced significantly serum sICAM-1 ([0%,6%], p=0.03), but no effects were observed on other tertiary markers. Lp-PLA₂ increased by 9% ([3%,16%], p=0.006) in response to the highest dose of DG-031 and there was comparable increase observed in LDL cholesterol (8% [4%,12%], p<0.001) that correlated with Lp-PLA₂. In contrast, the effects of the 2 lower doses (250 mg/day and 500 mg/day) on Lp-PLA₂ were not significant. Urine levels of LTE₄ increased by 27% in response to the highest dose of DG-031 ([15%,40%], p<0.001). Significant correlation was observed between the change of LTB₄ and MPO production (r=0.62 [0.51,0.70], p<0.001) within a period, also when considering only those taking drug where the range of these changes were larger. Correlation was also observed for these variables at baseline. DG-031 in the higher two dose groups reduced CRP by 16% ([−2%,31%], p=0.07) at 2 weeks, although this is not significant. Comparable effects on CRP were also observed after one week of therapy with the higher dose of DG-031 (38% [9%,57%], p=0.02) in the follow-up study. In addition, when examined 6 hours after intake of DG-031, plasma MPO levels dropped by 37% ([24%,47%], p<0.001) in the 250 mg t.i.d. dose group.

### Tests for Carry-Over Effects

A test for carry-over effects from the treatment phase to the placebo phase was performed as a two-sample t-test on the differences between visit 2 and 5 for patients on drug and placebo, respectively. The cohort taking drug consists of patients on 500 mg/day and 750 mg/day treatment and the placebo cohort includes patients on placebo from all 3 tracks. The resulting p-values and confidence intervals for the effect are given in Table 9 (data were not available for Lp-PLA₂ and N-tyrosine). No carry over effects were observed with LTB₄ and MPO. In contrast, marked carry over effects were observed for CRP and SAA, with reduction in CRP that was significant at the 5% level (p=0.02). SAA showed similar carry over effects that was slightly below this significance level (p=0.05).

TABLE 9
Test for carry-over effect for each study period

| Assay | p-value | Effect | 95% CI |
|---|---|---|---|
| CRP | 0.02 | −0.28 | [−0.52, −0.05] |
| Amyloid A | 0.05 | −0.14 | [−0.29, 0.00] |
| LTE₄ in urine | 0.48 | −0.06 | [−0.22, 0.10] |
| MCP-1 | 0.08 | 0.07 | [−0.01, 0.15] |
| MMP 9 | 0.56 | −0.04 | [−0.16, 0.09] |
| MPO in plasma | 0.28 | −0.11 | [−0.31, 0.09] |
| White blood cell count | 0.57 | −0.01 | [−0.06, 0.03] |
| LTB₄ in whole blood, corr. for wbc^{‡} | 0.45 | −0.10 | [−0.36, 0.16] |
| MPO in whole blood, corr. for wbc^{‡} | 0.93 | 0.01 | [−0.13, 0.15] |
| LTB₄ in whole blood, mass spec.^{§} | 0.45 | 0.19 | [−0.33, 0.71] |
| LTB₄ in whole blood, corr. for wbc, | 0.64 | 0.12 | [−0.40, 0.64] |
| m.s.^{§} | | | |

^{‡}corr. for wbc = corrected for white blood cell count
^{§}m.s. = mass spec. = mass spectrometry

A dose of 250 mg TID, reduced, on average, capacity for LTB₄ production by 30% ([0%,56%], p=0.02) at a steady state Cₘᵢₙ plasma concentration of 2-3 μg/ml. The 250 mg TID dose also reduced plasma levels of MPO in CAD patients by 37% ([24%,47%], p<0.001) and serum levels of CRP by 38% ([9%,57%], p=0.02). This dosing schedule was most efficacious in reducing capacity for LTB₄ production by ionomycin-activated neutrophils (ex-vivo). DG-031 also was observed to inhibit MPO release by activated leukocytes.

The degree of inhibition of LTB₄ production and MPO release in ionomycin-activated neutrophils (ex-vivo) was higher in patients on DG-031 therapy compared to placebo. The combined dose groups of 500 mg and 750 mg TDD exhibited about 26% reduction of capacity for LTB₄ production (p=0.003), and about 12% inhibition of MPO release (p=0.02). The observed inhibitory effects appeared to be dose-dependent. Capacity for LTB₄ production was reduced 13% in the 250 mg QD dose group (p=0.22), 18% in the 250 mg BID dose group (p=0.1), and 34% in the 250 mg TID dose group (p=0.008). The reduction in MPO release was seen only with the combined 500 mg and 750 mg TDD analysis. Blood samples for ex-vivo assays were drawn at steady state prior to patients receiving the next morning dose of DG-031.

### Example 4

### Persistent Effect of DG-031 on CRP Reduction

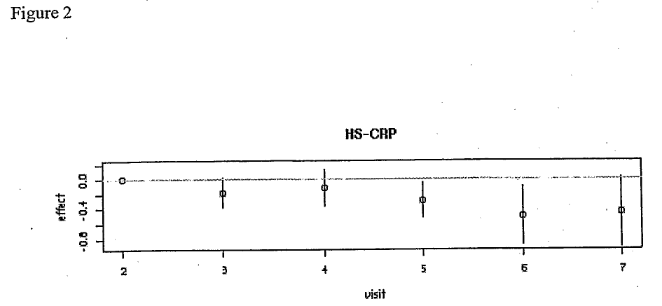

Relative to placebo therapy that had no effect, the 250 mg BID and 250 mg TID doses of DG-031 reduced serum CRP by 16% at 2 weeks, although this was not statistically significant (p=0.07). However, the effects on CRP were more pronounced at the end of the wash-out period (corresponding to the week 6 study visit) where the reduction in CRP was 25% (p=0.02) and persisted for another 4 weeks thereafter. DG-031 had qualitatively similar effects on SAA that amounted to a reduction of 15% on week 6 (p=0.05). See FIG. 2. This indicates that after an initial run in of DG-031 therapy at the dosing schedule of 250 mg TID, it may be possible after 2-4 weeks of treatment to reduce the maintenance dose of DG-031 to 250 mg BID or 250 mg QD so long as the reduced CRP levels continue to be maintained.

### Example 5

### In Vitro Effects of DG-031 on LTB4 Production and MPO Release

Neutrophils and other cells in human whole blood respond in vitro to activation by a calcium ionophore (e.g., ionomycin), with an increase in LTB₄ production and with coupled release of MPO stimulated in part by LTB₄ production in the assay. The effect of DG-031 on the capacity for production of LTB₄ in vitro after ionomycin stimulation was evaluated at concentrations ranging from 0.1 μM up to 240 μM.

The in vitro IC₅₀ for DG-031 is about 3 μM (1.1 μg/ml). Blood samples collected from the clinical study described in Example 2 were evaluated assuming that the ex vivo response profile in clinical samples parallels the in vitro response over the entire concentration range, 250 mg TID dosing which maintains concentrations above 2-3 μg/ml (˜7 μM) should maintain a 30% or greater reduction of capacity for LTB₄ production at all times. Peak concentrations of DG-031 with 250 mg TID dosing were about 10 μg/ml (28 μM) and should be associated with about 65% reduction of capacity for LTB₄ production. The typical patient's pharmacodynamic response was expected to fluctuate between 30% and 65% reduction of capacity for LTB₄ production over the course of a dosing interval. DG-031 also reduced release of MPO by neutrophils and other cells in human whole blood after ionomycin-stimulation. MPO release appears to be maximally inhibited in vitro once the concentration of DG-031 reached about 6 μM (˜2 μg/ml), which is the Cₘᵢₙ obtained with 250 mg TID dosing.

### Example 6

### Open Label, Dose Ranging Trials in CAD Patients (Study No. DG-031-203)

After completion of the double-blind study reported herein an open-label randomized study was conducted in an independent cohort but with same eligibility criteria and measuring several of the same biomarkers. This study included 75 patients in 3 tracks of equal size, each with a distinct dosing regimen of active drug, including the 250 mg TID dose. Each patient was on drug for 8 days; there was no placebo group. The primary objective of this study was to determine pharmacokinetic parameters of DG-031 in three different doses, as well as to assess the pharmacokinetic/pharmacodynamic relationship between DG-031, LTB₄ and MPO. Here we will refer to effects of treatment with DG-031 on CRP and MPO observed already following eight days of therapy with DG-031.

The additional phase II study was conducted to compare a 750 mg total daily dose administered in a BID or TID dosing regimen. The study was designed to provide pharmacokinetic information on repeated single daily dose administration of the 375 mg dose, as well as 375 mg BID. The calculated AUC₂₄ₕ was somewhat higher for 250 mg TID than for 375 mg BID. Although Cₘᵢₙ was comparable for the 375 mg BID and 250 mg TID, it was slightly higher for the TID dosing regimen.

The study also collected information on the dose-related effect of DG-031 on capacity for LTB₄ production (ex vivo), plasma MPO, and serum-CRP. Efficacy results for a total daily dose of 750 mg were similar to that demonstrated for the study described above. The pattern observed supported a concentration dependent pharmacologic effect of DG-031 on capacity for LTB₄ production measured ex vivo. The effects on LTB₄ production amounted to a 30% (p=0.016) and 25% (p=0.094) reduction with the 375 mg BID and 250 mg TID doses, respectively.

Dose dependent effects of DG-031 on the plasma MPO and serum CRP biomarkers also were observed that appeared to be related to Cₘᵢₙ where this was slightly higher for the TID dosing regimen. The 250 mg TID dose of DG-031 lowered plasma MPO levels as much as 37% (p<0.001) 6 hours after intake of drug, while 375 mg BID lowered plasma MPO levels by 24% (p=0.067) although this effect did not reach statistical significance.

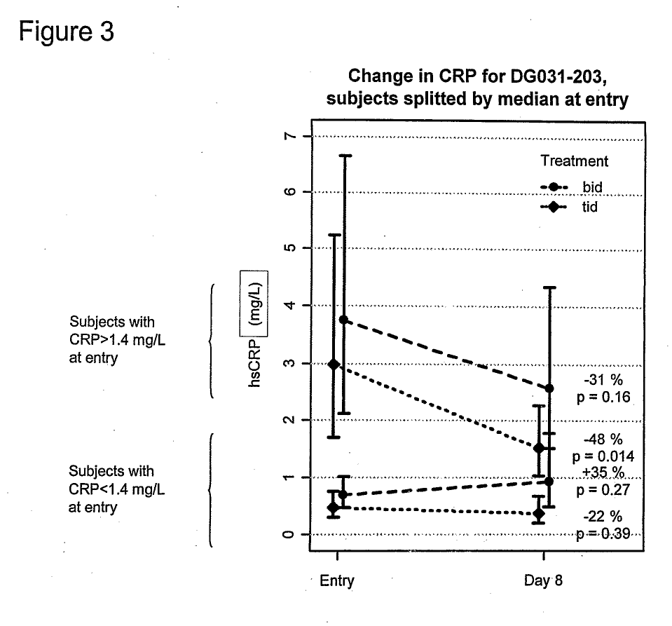

As shown in FIG. 3, the 250 mg TID dose also reduced serum CRP levels in all subjects by 38% (p=0.017) after one week of therapy, whereas the 375 mg BID dose had little effect on CRP in this limited study (−1% change, p=0.94). The 250 mg TID dose consistently caused a profound statistically significant reduction in CRP level. This effect was particularly observed in patients exhibiting high CRP levels at entry in the study (see FIG. 3). Dividing all subjects into those with high CRP above the median value for all patients at entry (CRP 1.4 mg/L) and those with low CRP below the median at entry, the 250 mg TID dosing schedule caused a 48% reduction in CRP levels in the high CRP group (FIG. 2), which was highly statistically significant (P=0.014) There was a trend for reduction of CRP by DG-031 administered as 375 mg BID (for the same daily dose of 750 mg), but this effect did not reach statistical significance (reduction of 31% p=0.16). Thus, the 250 mg TID DG-031 dosing schedule is a more preferable dosing schedule than 375 mg BID for reduction in CRP levels as it consistently caused reduction of CRP in all subjects treated and in the subgroup of patients with high CRP (>1.4 mg/L) at study entry.

Table 10 summarizes the data collected in clinical study no DG-031-201 (Example 3) and clinical study no. DG-031-203 (Example 5). As shown in Table 10, the 250 mg TID dosing schedule was clearly a superior treatment for reducing LTB4, MPO and CRP levels. The 250 mg TID dosing schedule consistently reduced LTB4, MPO and CRP levels and these reductions were statistically significant. Therefore the 250 mg TID dosing schedule is likely to provide the greatest clinical benefit for therapy with DG-031.

TABLE 10

| | Total Daily Dose | | | | |
|---|---|---|---|---|---|
| | 250 mg | 375 mg | 500 mg | 750 mg (Study | 750 mg |
| | (Study 201) | (Study 203) | (Study 201) | 201 & 203) | (Study 203) |
| Dose and | 250 mg | 375 mg | 250 mg | 250 mg | 375 mg |
| regimen | QD | QD | BID | TID | BID |
| # of | 64 | 25 | 64 | 63 (St. 201) | 25 |
| Subjects | | | | 25 (St. 203) | |
| LTB4 | −13% | −16% | −18% | −34% (St. 201) | −30% |
| production | NS | 2 hours | NS | p = 0.008 | 2 hours |
| | | NS | | −25% (St. 203) | p = 0.016 |
| | | | | p < 0.001 | |
| MPO in | ND | No effect | ND | −37% (St. 203) | −24% |
| plasma | | 6 hours | | 6 hours | 6 hours |
| | | | | P < 0.001 | NS |
| CRP | −6% | −28% | −24% | −13% (St. 201) | No effect |
| | 2-6 wks | 1 wk | 2-6 wks | 2-6 wks, NS | observed |
| | NS | p = 0.021 | p = 0.016 | 37% (St. 203) | |
| | | | | 1 wk, p = 0.07 | |

In addition, the reduction in both MPO and CRP biomarkers was additive to the reduction already achieved by statin therapy, since essentially all patients were on stable doses of cholesterol lowering drugs (statins) prior to initiation of DG-031 dosing. Therefore, treatments that coadminister the 250 mg TID dosing schedule and a statin are contemplated by the invention.

### Example 7

### Pharmacokinetic Analysis of DG-031 Dosing Schedules

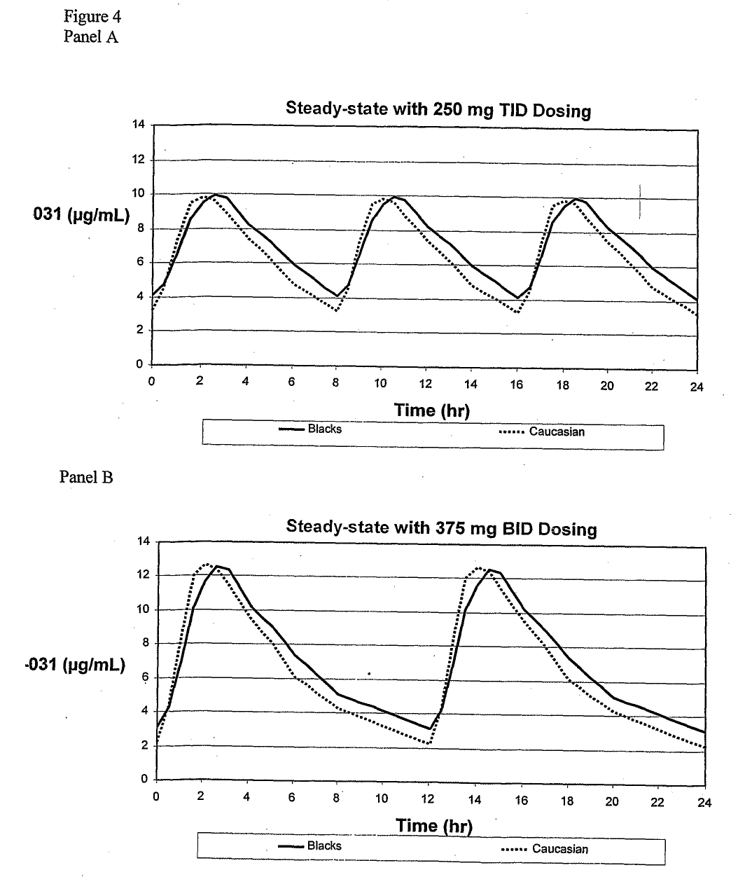

When summarizing the two clinical studies, DG-031-201 and DG-031-203, reasonable estimates of the steady state concentrations for the DG-031 dosing schedules of 375 mg BID and 250 mg TID were provided. DG-031 pharmacokinetics in humans is determined by a relatively short half-life that accounts for most of the administered drug, and a somewhat longer half-life that associated with a smaller fraction of the administered drug. Therefore, by gross appearances steady-state peak and trough values do not appear to be greatly affected by the choice of once daily (QD), twice daily (BID) or three times daily (TID) dosing. However, the rapid and slow phases of elimination appear to play a critical role in the selection of dosing frequency for optimal efficacy, when the efficacy is related to maintaining concentrations above a threshold value. The dominant half-life of the DG-031 is about 2.5 to 3 hours. For drugs with such a short half-life, a dosing schedule of 250 mg TID is more effective for maximizing therapeutic effectiveness when effectiveness requires maintaining concentrations above a minimum values. For a drug within a 2.5 hour half-life, TIED dosing yields a peak plasma concentration about of 11 μg/mL which is exposed to be required to keep steady-state concentration above 3 μg/ml for the entire dosing interval. Maintaining the same minimum concentrations with a BID dosing schedule requires peak concentration to reach an estimated 30 μg/mL as shown in FIG. 4. Based on these calculations, the daily amount of drug that must be administered using the BID dosing schedule is about twice that needed when compared to a TID dosing schedule.

With DG-031, part of the administered dose has an eliminated half-life longer than 2.5 3.0 hours. The fraction of the dose with the slower half-life of around 10-15 hours accounts for only about 25% of the total exposure, but leads to enough accumulation at steady-state that a trough concentration of 3 μg/mL can be maintained with BED doses that produce peak concentrations of only 14 μg/mL, or with TID doses that produce peak concentrations of just 7-8 μg/mL. Maintaining the minimum concentration using TIED dosing requires only about 70% of the drug that is required for a BID schedule. TID dosing is a more efficient process in terms of drug utilization when the goal is maintaining concentrations above a minimum threshold. It has the added advantage of reducing a patient's total exposure to the drug.

### Example 8

### Open-Label, Randomized Study on High Doses of DG-031 in Patients with CAD (Study DG-031-204)

An additional single site, open label study was carried out to obtain biomarker and safety data on additional DG-031 doses (i.e., 1000 mg and 1500 mg). This study examined the safety, tolerability and dose-related effects of DG-031 on inflammatory biomarkers including: LTB4, hsCRP, and MPO. A total of 119 patients with a prior history of CAD and MI were randomized to receive either 1000 mg DG-031 (500 mg BID) or 1500 mg DG-031 (500 mg TID) daily for 4-6 weeks. Treatment with DG-031 in addition to the subject's standard care, medication, and treatment, was prescribed by the patient's cardiologist. A majority of the subjects received statin therapy before, during and after the study. Follow-up assessments were performed 2 weeks after the end of study drug administration.

The biomarkers were analyzed as described in Example 1. Statistical analysis of the data was carried out using a one-sample t-test on change from baseline with a two-sided nominal significance level of 0.050. Biomarker concentrations were log-transformed and a simple last observation carried forward (LOCF) scheme was used to impute missing observations.

A total of 119 patients were included in the analysis. The following table summarizes the demographic characteristics of the patients by treatment group.

TABLE 11
Study DG-031-204 patient demographics (mean and sd)

| | DG-031 | DG-031 |
|---|---|---|
| | 500 mg BID | 500 mg TID |
| N | 60 | 59 |
| Gender | 9 F/51 M | 10 F/49 M |
| Age (yrs) | 65.7 (8.5) | 64.9 (8.2) |
| Body Weight (kg) | 91.4 (16) | 89.2 (13) |
| LDL (mmol/L) | 2.6 (0.75) | 2.9 (0.87) |
| Glucose (mmol/L) | 6.0 (1.7) | 5.7 (1.3) |
| HapA/HapK/HapC+ | 50% | 40.7% |
| HapA+ | 28.3% | 23.7% |
| HapK+ | 21.7% | 20.3% |
| HapC+ | 21.7% | 22.0% |

### Biomarker Analysis

Serum was collected weekly for determination of LTB4 concentration in response to the treatment with DG-031. Whole blood was also collected at the end of treatment in a subset of patients for determination of LTB4 concentrations following ionomycin stimulation as described in Example 1. The mean change from baseline after 4 weeks of treatment is listed in the Table 12. Serum LTB4 concentrations decreased significantly from baseline. The decrease in serum LTB4 was evident at one week (1st measurement) and persisted throughout the dosing phase. Stimulated LTB4 also decreased significantly. A preliminary exposure-response analysis of data from studies DG-031-201 and DG-031-203 (Examples 3 and 6, respectively) supported a dose related decrease in (stimulated) LTB4 levels. Stimulated LTB4 effects measured in study DG-031-204, as shown in the raw data set out in Table 12, appear consistent with projections from the exposure-response analysis based on data from studies DG-031-201 and DG-031-203. Data presented previously from studies DG-031-201 and DG-031-203 is presented as raw data in Table 13 below. The raw data are the observations at any specified time point during the study.

TABLE 12
LTB₄ percent change from baseline after 4 weeks of DG-031
administration

| | 1000 mg | 1500 mg |
|---|---|---|
| | (500 mg BID) | (500 mg TID) |
| | mean response | mean response |
| | p, comparing to | p, comparing to |
| Measure | baseline n | baseline n |
| Serum LTB₄ | −73% | −50% |
| (% change from | p: 0.0024 | p: 0.06 |
| baseline) | n = 16 | n = 13 |
| Stimulated LTB₄ | −44% | −28% |
| (% change from | p: <0.001 | p: <0.001 |
| baseline, whole blood, | n = 35 | n = 36 |
| WBC corrected, 15 | | |
| minute) | | |

TABLE 13
LTB4 percent change from baseline in Icelandinc Haplotype
Positive CAD patients, studies DG-031--201 and DG-031--203
(exposure of 1 week to 1 month)

| | Total daily dose | | | | |
|---|---|---|---|---|---|
| | 250 mg | 375 mg | 500 mg | 750 mg | 750 mg |
| | (−201) | (−203) | (−201) | (−201 & −203) | (−203) |
| Dose and | 250 mg | 375 mg | 250 mg | 250 mg | 375 mg |
| regimen | QD | QD | BID | TID | BID |
| # Subjects | 64 | 25 | 64 | 63 (−201) | 25 |
| | | | | 25 (−203) | |
| LTB4 | −13% | −16% | −18% | −34% (−201) | −30% |
| production | NS | 2 hrs | NS | p = 0.008 | 2 hrs |
| | | NS | | −25% (−203), | p = 0.016 |
| | | | | 2 hrs, NS | |

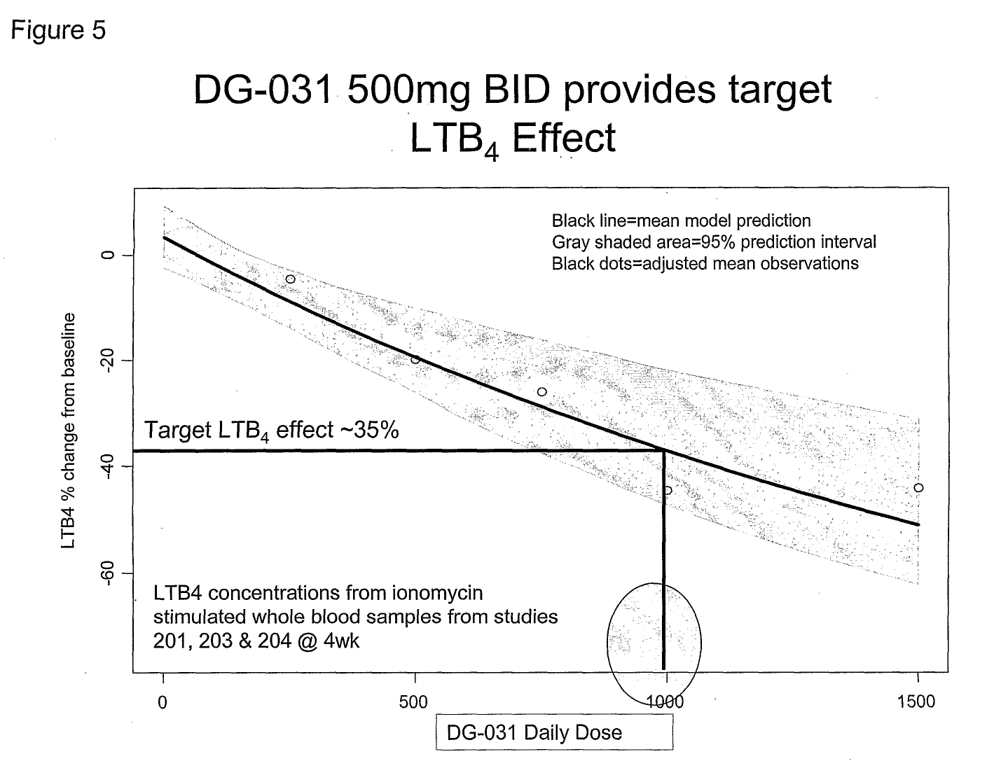

The data from lower dose clinical studies suggested that with proper dosing, LTB4 production in MI patients could be reduced approximately 35% from baseline to approach the level in the control subjects. Administration of DG-031 500 mg BID reduced LTB4 levels to the target 35% reduction, as measured in the ionophore-stimulation assay described in Example 1. The estimated DG-031-induced reduction in stimulated LTB4 levels was based on all the relevant data observed in the DG-031-201 and DG-031-203 studies. This estimated reduction in LTB4 level is dose dependent as shown in Table 14. The control baseline was 7.8%. The data presented in Table 14 are the mean and the 5^{th} and 95^{th} percentiles based on the mean model prediction (the 90% confidence interval on the estimated LTB4 response). This data is also presented in FIG. 5.

TABLE 14
Estimated Percent LTB4 Reduction by DG-031 Daily Dose

| Daily Dose | 5^{th} Percentile | Mean | 95^{th} Percentile |
|---|---|---|---|
| 0 | −1.5 | 3.3 | 8.1 |
| 250 | −13.1 | −8.7 | −3.8 |
| 500 | −24.8 | −19.4 | −11.7 |
| 750 | −35.7 | −28.8 | −18.8 |
| 1000 | −45.3 | −37.1 | −24.5 |
| 1250 | −53.5 | −44.5 | −29.9 |
| 1500 | −60.4 | −50.9 | −35.2 |

Plasma MPO concentration was measured weekly. MPO measurements in ionomycin-stimulated whole blood were also measured in a subset of patients at 4 weeks. No changes from baseline in plasma MPO concentrations were noted. Stimulated whole blood MPO concentrations decreased following DG-031 administration, as summarized in Table 15.

TABLE 15
MPO percent change from baseline after 4 weeks of DG-031
administration

| | 1000 mg | 1500 mg |
|---|---|---|
| | (500 mg BID) | (500 mg TID) |
| | mean response | mean response |
| | p, comparing to | p, comparing to |
| Measure | baseline n | baseline n |
| Serum MPO | +3% | −12% |
| (% change from | p: 0.73 | p: 0.097 |
| baseline) | n = 55 | n = 55 |
| Stimulated MPO from | −19% | −29% |
| whole blood | p: 0.0082 | p: 0.0033 |
| (% change from | n = 36 | n = 34 |
| baseline, whole blood, | | |
| WBC corrected, 60 | | |
| minute) | | |

Additionally, hsCRP was measured weekly and tended to decrease in the 1000 mg treatment group. However, hsCRP did not change significantly in the 1500 mg treatment group. The mean change from baseline after 4 weeks of treatment was 20% reduction (p=0.046; n=52) in the 1000 mg treatment group and 3% reduction (p=0.77; n=58) in the 1500 mg treatment group. A preliminary exposure-response analysis of data from studies DG-031-201, DG-031-203 (Examples 3 and 6), and this study (DG-031-204) did not support a dose related effect on hsCRP.

CD40L was measured at 2-week intervals. CD40L was significantly decreased 2 and 4 weeks during treatment. After 4 weeks, there was a 22% (p<0.001; n=55) reduction in CD40L in the 500 mg BID group and a 21% (p<0.001; n=57) reduction in the 500 mg TID group. CD40L levels remained suppressed at the 2 week follow up visit. A preliminary exposure-response analysis of data from studies DG-031-201, DG-031-203, and DG-031-204 did not support a dose related decrease in CD40L.

Prostaglandin F (PGF) was measured at 2-week intervals. No significant trends in PGF were observed after 4 weeks of treatment.

### Safety Analysis

A total of 5 patients reported 8 serious adverse events (SAE). One death was reported during the trial. All SAE's were consistent with underlying CAD disorders (see Table 16). A total of 88 of 119 patients reported 203 adverse events. The most commonly reported adverse events (and number of cases reported) included: cold (27), headache (13), constipation/diarrhea (6/8), and vertigo (5).

TABLE 16
Serious Adverse Events

| DG-031 | Patient | |
|---|---|---|
| Treatment | Number | SAE Description |
| 1500 mg | 3055 | Congestive heart failure |
| 1000 mg | 3088 | Myocardial infarction |
| 1000 mg | 3180 | Unstable angina |
| 1000 mg | 3200 | Myocardial infarction, |
| | | Cardiac arrest, |
| | | Subarachnoid hemorrhage |
| 1000 mg | 3234 | Angiography, |
| | | Repair of Carotid artery |

LDL-C was measured weekly after administration of DG-031 500 mg BID or 500 mg TID. The mean change from baseline after 4 weeks of treatment was an increase of 9% (p=0.004; n=52) for 500 mg BID and an increase of 9% (p<0.001; n=58) for the 500 TID group. A significant increase in LDL-C was observed with DG-031 administration as shown in the raw data in Table 17. The LDL-C effect appeared to increase over time and tended toward baseline values at the 2-week follow up visit. A preliminary exposure-response analysis of data from studies DG-031-201, DG-031-203, and DG-031-204 supported a dose related increase in LDL-C.

TABLE 17
LDL-C percent change from baseline after 4 weeks of DG-031
administration

| Dose | 5% | Mean | 95% |
|---|---|---|---|
| 0 | −1.6 | −0.4 | 0.7 |
| 250 | 1.2 | 2.3 | 3.3 |
| 500 | 3.7 | 5.0 | 6.0 |
| 750 | 5.9 | 7.7 | 9.0 |
| 1000 | 8.1 | 10.3 | 12.1 |
| 1250 | 10.3 | 13.0 | 15.2 |
| 1500 | 12.4 | 15.7 | 18.3 |

Liver function tests (AST, ALT, GGT) were assessed weekly during study drug administration and two weeks after dosing. No adverse trends in mean liver function tests (AST, ALT, GGT) were noted. Serum creatinine (Cr) was measured weekly during study drug administration and two weeks after dosing. In both the 500 mg BID and 500 mg TID treatment groups, mean serum Cr concentrations increased 4-5% relative to baseline levels. At the 2-week follow up visit, mean serum Cr returned to baseline in the cohort treated for 4 weeks, but remained elevated in the group treated for 6 weeks. Creatine kinase (CK) measurements were obtained weekly during study drug administration and two weeks after dosing. No adverse trends in mean CK concentrations were noted.

### Example 9

### Pharmokinetic Analysis Based on Various Clinical Studies

Preliminary population pharmacokinetic (PK) analysis was also carried out based on clinical studies DG-031-201 and DG-031-203. Based on visual inspection of the individual and mean time course of DG-031 concentrations, a simple one compartmental disposition model following first order oral absorption, and an absorption lag time was fit to the data. A mixed effects modeling approach was used. The pharmacokinetic model parameters (fixed effects) included oral clearance (CL), distribution volume (V), oral absorption (ka), and absorption lag (tiag). The statistical model includes inter-individual random effects as well as a residual error term. All inter-subject random effect parameters (it's) and residual error terms (&) are assumed to be normally distributed with mean of zero and variance o2 and a2, respectively. Random effects on all pharmacokinetic model parameters were explored. Maximum likelihood estimates of the structural (0's) and statistical model parameters were estimated using an approximation to the mixed effect log-likelihood as implemented in the NONMEM program (NONMEM Version Vi).

The influence of patient characteristics (covariates) on pharmacokinetic parameters was explored. Age, gender, body weight, and Cr_{CL} (creatinine clearance) were included in the model as covariates if warranted.

Model selection will be done on the basis of the log-likelihood criterion (p<0.05) and visual inspection of goodness-of-fit plots. The difference in −2 times the log of the likelihood (−2LL) between a full and reduced model is asymptotically χ² distributed with degrees of freedom equal to the difference in number of parameters between the two models. For instance, a decrease of more than 3.84 in −2LL is considered significant at the p<0.05 level for 1 additional parameter. Standard errors of the parameter estimates are approximated using the asymptotic covariance matrix.

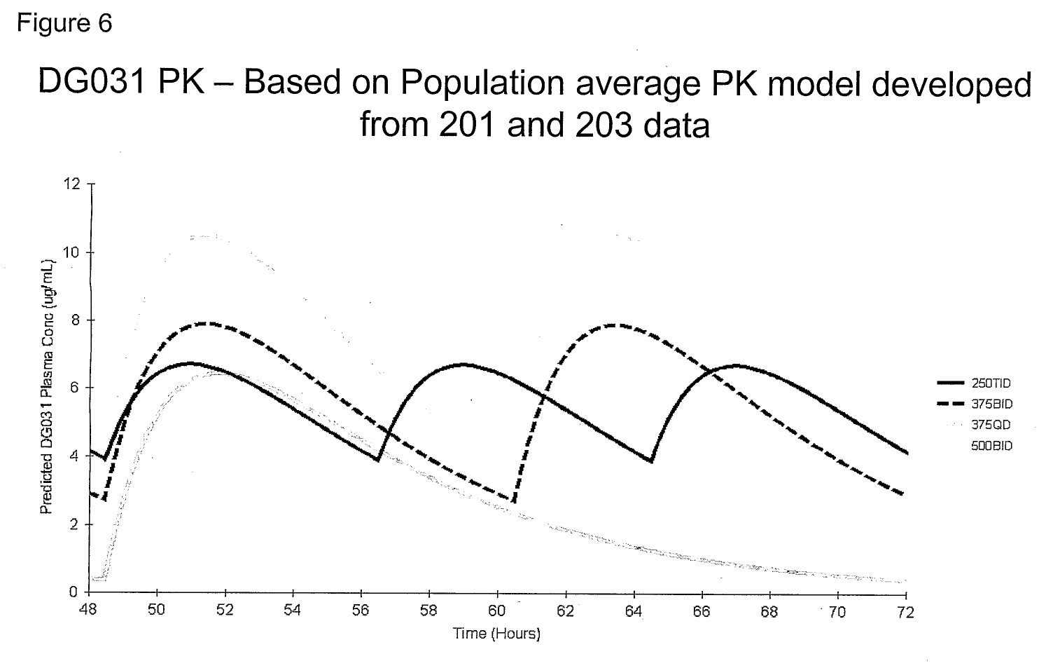

The PK parameters were well established. There was a linear PK from 250 mg to 1500 mg administered daily. There was 12% accumulation with BID dosing. The oral CL was 5.86 L/hr for a 90 kg patient and 30% inter-subject coefficient of variation (CV %), V=32 L and 44% inter-subject CV %. Body weight had minor effects on CL and there was no effect of age, gender or Creatinine clearance (Cr_{CL}) on DG-031 CL. FIG. 6 displays the DG-031 PK based on population average PK model developed from the DG-031-201 and DG-031-203 clinical studies.

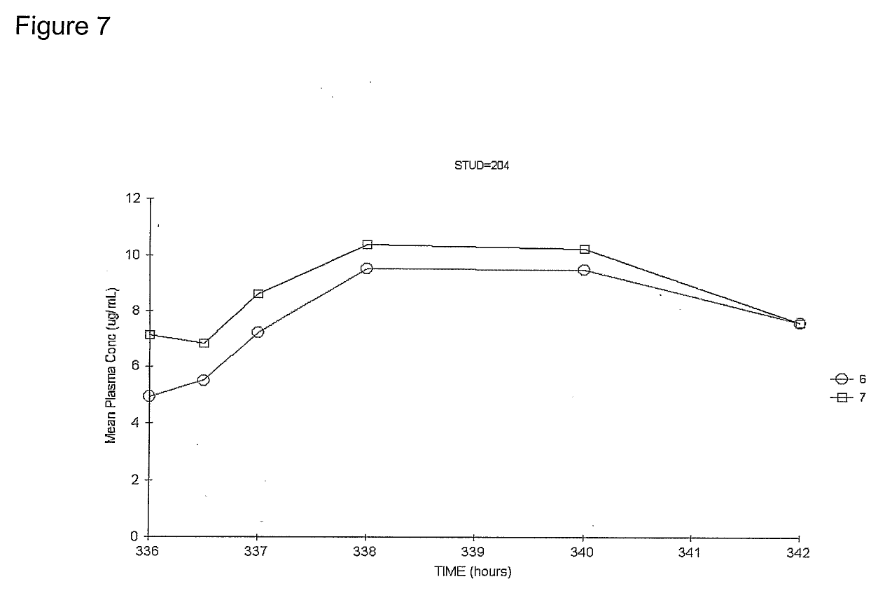

Blood samples were collected for determining DG-031 concentrations in a subset of patients (n=22). Samples were collected just prior to dosing and at 0.5, 1, 2, 4, and 6 hours following administration of DG-031 500 mg BID or TID. DG-031 concentrations up to 20.3 μg/mL were observed in this study. The geometric mean DG-031 steady-state concentrations are set out in Table 18 below and are displayed graphically in a concentration-time profile in FIG. 7.

Exposure-response analysis of biomarker data collected in MI patients (studies DG-031-201, DG-031-203, and DG-031-204) suggest that DG-031 administered 500 mg BID (1000 mg daily) will give an average drug exposure of 7.6 μg/mL associated with a 37% (90% prediction interval; −45% to −24%) reduction in stimulated LTB₄ production. The pharmacokinetic characteristics of the tested DG-031 formulation result in an observed mean peak concentrations of 8.8 μg/mL and mean trough concentrations of 4.3 μg/mL. (Table 18) The characteristics of the tested formulation leads to prolonged drug absorption that minimizes the peak to trough fluctuation in DG-031 plasma concentration and LTB4 response.

TABLE 18
Study DG-031-204 Steady State DG-031 Exposures

| | Time Post | | Geometric | | |
|---|---|---|---|---|---|
| | Dose | | Mean | Min | Max |
| Regimen | (hr) | n | (μg/mL) | (μg/mL) | (μg/mL) |
| 500 mg BID | 0 | 12 | 4.3 | 1.1 | 9.1 |
| 500 mg BID | 0.5 | 12 | 4.7 | 2.2 | 15.5 |
| 500 mg BID | 1 | 12 | 6.1 | 2.3 | 18.7 |
| 500 mg BID | 2 | 12 | 8.6 | 2.9 | 17.7 |
| 500 mg BID | 4 | 12 | 8.8 | 4.2 | 20.3 |
| 500 mg BID | 6 | 12 | 7.0 | 3.3 | 14.9 |
| 500 mg TID | 0 | 9 | 6.9 | 4.1 | 8.8 |
| 500 mg TID | 0.5 | 9 | 6.8 | 5.4 | 8.3 |
| 500 mg TID | 1 | 9 | 8.5 | 5.6 | 11.3 |
| 500 mg TID | 2 | 9 | 10.1 | 6.5 | 15.1 |
| 500 mg TID | 4 | 9 | 10.0 | 7.2 | 13.4 |
| 500 mg TID | 6 | 9 | 7.4 | 4.4 | 10.3 |

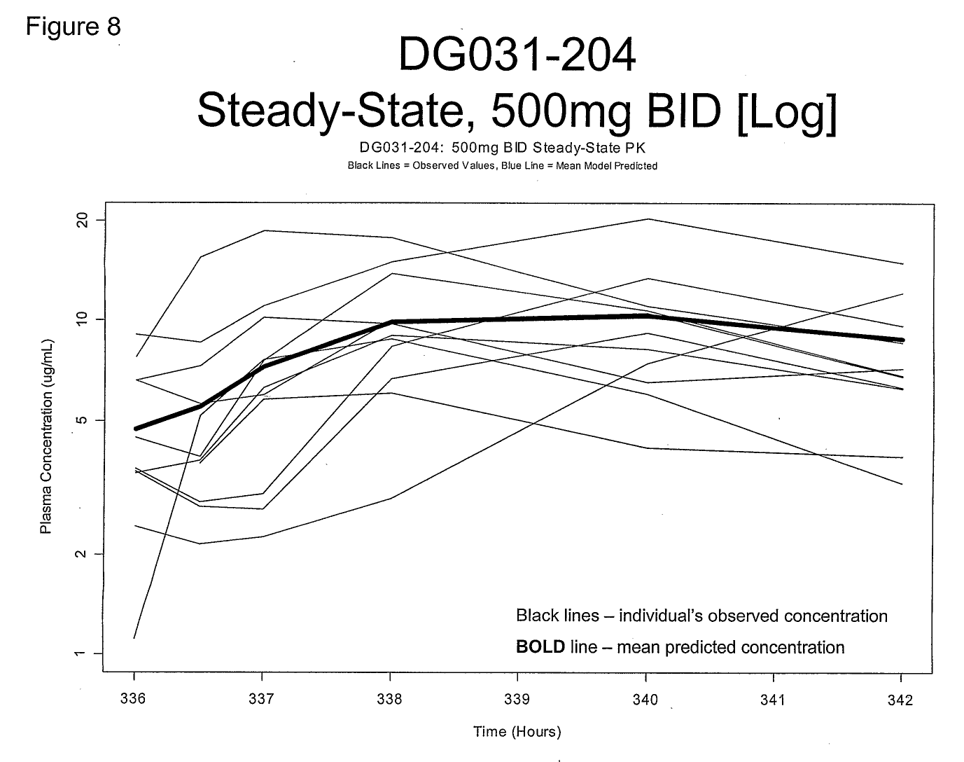

This analysis determined that the estimated DG-031 terminal elimination half-life in MI patients is approximately 4 hours. Given this half-life drug concentrations, DG-031 concentrations would be expected to fall approximately 8-fold over a 12 hour dosing interval (e.g., 3 half-lives). However, due to a lag time in drug absorption and a prolonged absorption phase following oral administration of the tested DG-031 tablet, the observed peak to trough fluctuation is only 2-3 fold (see FIG. 8) over a 12 hour period under a BID dosing regimen. Consequently, the tested DG-031 formulation is well suited for twice daily dosing (BID).

1. A method of treating or preventing an inflammatory condition or disease in a human comprising administering doses of DG-031, or a pharmaceutically acceptable salt, ester, or pro-drug thereof to a human subject in need of treatment or prevention of an inflammatory condition or disease, according to a dosing schedule that is effective to maintain a steady state DG-031 plasma concentration in a range of 6 μM to 31 μM in said human.

2. (canceled)

3. A method of treating or preventing an inflammatory condition or disease in a human comprising administering doses of DG-031, or a pharmaceutically acceptable salt, ester or prodrug thereof, to the human according to a dosing schedule that is effective to maintain a steady state DG-031 plasma concentration of at least 6 μM; and provide a peak/trough (Cₘₐₓ/Cₘᵢₙ) plasma concentration ratio of less than 5.

4. (canceled)

5. The method according to claim 138, wherein the doses are formulated for oral administration and administered orally.

6. The method according to claim 1, wherein the dosing schedule is no more than three times per day.

7. The method according to claim 1, wherein the dosing schedule is two times per day.

8. The method according to claim 1, wherein the doses and dosing schedule are effective to maintain a steady state DG-031 plasma concentration of at least 8 μM.

9-13. (canceled)

14. The method according to claim 1, wherein the doses and dosing schedule are effective to maintain a steady state DG-031 plasma concentration below 30 μM in said human.

15-23. (canceled)

24. The method according to claim 1, wherein the doses and dosing schedule are effective to provide a peak:trough DG-031 plasma concentration ratio of less than 4.

25-28. (canceled)

29. The method according to claim 1, wherein the inflammatory disease or condition is a cardiovascular disease or condition.

30. The method or use according to claim 29, comprising a step, prior to the administering step, of selecting a human at risk for myocardial infarction to receive the doses of DG-031.

31. The method according to claim 29, wherein the selecting comprises determining a level of an inflammatory marker in a human subject and selecting a subject with an elevated measurement of the marker.

32. (canceled)

33. The method according to claim 30, wherein the selecting comprises selecting a human who has suffered at least one myocardial infarction or selecting a human with a genetic predisposition to increased risk for myocardial infarction.

34. (canceled)

35. The method according to claim 33, wherein the genetic predisposition comprises presence of a polymorphism or haplotype in the human that correlates with increased risk for MI, wherein the polymorphism or haplotype is in a gene selected from the group consisting of FLAP, LTA4-H, and 5-LO.

36. The method according to claim 1, wherein the human is female.

37. The method according to claim 29, wherein the selecting comprises determining age and selecting a human that is at least 40 years old.

38-40. (canceled)

41. The method according to claim 1, further comprising determining if a human has a race that includes black African ancestry, and selecting for dosing with DG-031 a human with a race that includes black African ancestry.

42. The method according to claim 41, comprising determining if a human has a race that includes European ancestry, and selecting a human with a race that includes European and African ancestry.

43. The method according to claim 138, wherein the doses and dosing schedule are effective to achieve a reduction of leukotriene B4 (LTB4) of at least 30% within one week and maintain said reduction with continued administration of doses according to the dosing schedule.

44-46. (canceled)

47. The method use according to claim 138, wherein the doses and dosing schedule are effective to achieve a reduction of serum myeloperoxidase (MPO) of at least 15% within one week and maintain said reduction with continued administration of doses according to the dosing schedule.

48. The method according to claim 47, wherein the MPO is measured from a whole blood sample following contacting the whole blood sample with a calcium ionophore.

49-61. (canceled)

62. The method according to claim 1, wherein the doses are in a range of 1039 to 1385 micromoles of the DG-031 (375 to 500 mg) or the salt or ester thereof, and wherein the dosing schedule is 2 times per day.

63. The method according to claim 62, wherein the dose is 1385 micromoles of the DG-031 (500 mg) or the salt or ester thereof.

64. The method according to claim 1, wherein the dose is administered in a sustained release dosage form and the dosing schedule is twice per day.

65. The method according to claim 1, wherein the dose is administered in a sustained release dosage form and the dosing schedule is once per day.

66. The method according to claim 65, wherein the dose is in a range of 693-2770 micromoles of the DG-031 (250-1000 mg) or the salt or ester thereof.

67. The method according to claim 66, wherein the dose is 2770 micromoles of the DG-031 (1000 mg) or the salt or ester thereof.

68-71. (canceled)

72. The method according to claim 138, wherein the administering is performed for at least 90 days.

73-75. (canceled)

76. A controlled release formulation for oral administration to a human comprising DG-031, or a salt, ester, or prodrug thereof, in an amount effective to provide a mean minimum plasma concentration (Cₘₐₓ) of DG-031 in the range of 6 μM to 15 μM and a mean maximum plasma concentration of DG-031 in the range of 10 μM to 31 μM after repeated oral administration every 12 hours through steady state conditions.

77-79. (canceled)

80. A controlled release formulation according to claim 76 that is effective to provide a mean maximum plasma concentration of DG-031 in the range of 15 μM to 31 μM after repeated oral administration every 12 hours through steady state conditions.

81-83. (canceled)

84. A controlled release formulation according to claim 76 that is effective to provide a mean minimum plasma concentration of DG-031 in the range of 8 μM to 15 μM and a mean maximum plasma concentration of DG-031 in the range of 20 μM to 28 μM after repeated oral administration every 12 hours through steady state conditions.

85-87. (canceled)

88. A controlled release formulation according to claim 76, wherein the ratio of the mean maximum plasma concentration (peak) and the mean minimum plasma concentration (trough) of DG-031 after repeated oral administration every 12 hours through steady state conditions is less than 5.

89-93. (canceled)

94. A controlled release formulation according to claim 76, containing from 693 to 1385 micromoles of DG-031 (250 mg to 500 mg) or the salt or ester thereof.

95-96. (canceled)

97. A controlled release formulation according to claim 76, containing from 2077 to 2770 micromoles of DG-031 (750 mg to 1000 mg) or the salt or ester thereof.

98. A controlled release formulation according to claim 76, containing from 2770 to 4155 micromoles of DG-031 (1000 mg to 1500 mg) or the salt or ester thereof.

99. A controlled release formulation according to claim 76, wherein the mean maximum plasma concentration of DG-031 is detectable 4 to 6 hours after administration.

100-101. (canceled)

102. A controlled release formulation for oral administration to a human comprising DG-031, or a salt or ester or prodrug thereof, in an amount effective to provide a mean minimum plasma concentration of DG-031 from 6 μM to 15 μM and a mean maximum plasma concentration of DG-031 from 20 μM to 31 μM after repeated administration every 24 hours through steady state conditions.

103-104. (canceled)

105. A controlled release formulation according to claim 102 that is effective to provide a mean maximum plasma concentration of DG-031 in the range of 15 μM to 31 μM after repeated oral administration every 24 hours through steady state conditions.

106-111. (canceled)

112. A controlled release formulation according to claim 102, wherein the ratio of the mean maximum plasma concentration (peak) and the mean minimum plasma concentration (trough) of DG-031 after repeated oral administration every 24 hours through steady state conditions is less than 5.

113-116. (canceled)

117. A controlled release formulation according to claim 102 comprising from 1108 to 2770 micromoles of DG-031 (400 mg to 1000 mg of DG-031) or the salt or ester thereof.

118-119. (canceled)

120. A controlled release formulation according to claims claim 102, comprising from 2077 to 2770 micromoles of DG-031 (750 mg to 1000 mg of DG-031) or the salt or ester thereof.

121. A controlled release formulation according to claim 102, comprising from 2770 to 4155 micromoles of DG-031 (1000 mg to 1500 mg of DG-031) or the salt or ester thereof.

122. A controlled release formulation according to claim 102, wherein the mean maximum plasma concentration of DG-031 is detectable 10 to 12 hours after administration.

123-125. (canceled)

126. A controlled release DG-031 formulation according to claim 76, wherein the formulation is a solid tablet.

127. A controlled release DG-031 formulation of claim 126 wherein the solid tablet comprises a film coating.

128. A controlled release DG-031 formulation of claim 127 wherein the film coating reduces dissolution of the tablet in stomach acid with a pH less than 5.0.

129. A controlled release DG-031 formulation of claim 128, wherein the film coating reduces dissolution of the tablet in stomach acid with a pH less than 5.0, and wherein the film coating allows for tablet dissolution at a pH greater than 6.0.

130. A controlled release DG-031 formulation of claim 129, wherein the film coating is Eudragit L 100, Eudragit S100, Eudragit L 100-55, Colorcon Surlease, or FMC Aquacoat CPD.

131. A controlled release oral dosage formulation according to claim 76, comprising an effective amount of a controlled release matrix and a pharmaceutically acceptable diluent, wherein the controlled release matrix is selected from the group consisting of methylhydroxypropylcellulose, hypomellose phthalate polymer, ethylcellulose, polymethacrylate, hydroxypropyl methylcellulose acetate succinate, cellulose acetate phthalate (CAP) polymer and acrylic resin.

132. A method of treating or preventing an inflammatory condition or disease in a human comprising administering initial doses of DG-031, or a pharmaceutically acceptable salt or ester or prodrug thereof, according to an initial dosing schedule that is effective to maintain a steady state DG-031 plasma concentration in a range of 6 μM to 31 μM in said human, continuing administration of the initial doses of DG-031 according to the initial dosing schedule for a time effective to cause a reduction of LTB4 of at least 30%, and administering a maintenance dose of DG-031, or a pharmaceutically acceptable salt or ester or prodrug thereof, according to a maintenance dosing schedule after the reduction in LTB4, wherein the maintenance dose of DG-031 and the maintenance dosing schedule are effective to maintain a reduction of serum LTB4 of at least 30%.

133. (canceled)

134. The method of claim 132, wherein the total daily administration according to the maintenance doses and dosing schedule is at least 25% less than the total daily administration according to the initial doses and dosing schedule.

135. The method of claim 134, wherein the maintenance doses are 693 micromoles of the DG-031 (250 mg) or the salt or ester or prodrug thereof, and the maintenance dosing schedule is two times per day.

136. The method of claim 134, wherein the maintenance doses are 693 micromoles of the DG-031 (250 mg) or the salt or ester or prodrug thereof and the maintenance dosing schedule is once per day.

137. The method of claim 134, wherein the administering of the initial doses of DG-031 continues for 2 weeks.

138. A method of reducing leukotriene production in a human comprising administering to the human a composition that comprises a leukotriene synthesis inhibitor of the following formula or a pharmaceutically acceptable salt, ester, or prodrug thereof: 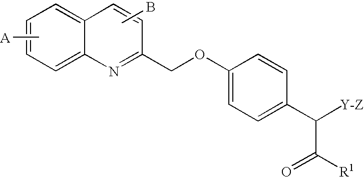
or pharmaceutically acceptable salt thereof, wherein R¹ represents a group of the formula: 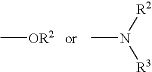
R² and R³ are identical or different and represent hydrogen, lower alkyl, phenyl, benzyl or a group of the formula: 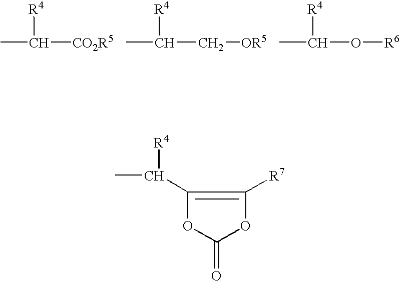
R⁴ represents hydrogen, lower alkyl, phenyl or benzyl, which can optionally be substituted by hydroxyl, carboxyl, lower alkoxycarbonyl, lower alkylthio, heteroaryl or carbamoyl, R⁵ represents hydrogen, lower alkyl, phenyl or benzyl, R⁶ represents a group of the formula —COR⁵ or —CO²R⁵, R⁷ represents hydrogen, lower alkyl or phenyl, Y represents a group of the formula: 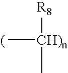
wherein R⁸ represents hydrogen, lower alkyl or phenyl and n denotes a number of 0 to 5, Z represents norbornyl, or represents a group of the formula: 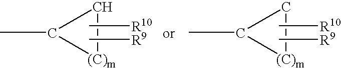
wherein R⁹ and R¹⁰ are identical or different and denote hydrogen, lower alkyl or phenyl, or R⁹ and R¹⁰ can together form a saturated carbocyclic ring having up to 6 carbon atoms and m denotes a number from 1 to 6, and A and B are identical or different and denote hydrogen, lower alkyl or halogen;
wherein the human is administered daily doses of 1000-1500 mg according to a dosing schedule effective to achieve a reduction of leukotriene B4 (LTB4) of at least 25% within one week and maintain said reduction with continued administration of doses according to the dosing schedule.

139. The method of claim 138, wherein the dose is formulated for sustained release.

140. The method of claim 138, wherein the daily dose is administered as 500 mg doses twice or three times per day.

141. The method according to claim 138, wherein the leukotriene synthesis inhibitor comprises BAY-X-1005 (DG-031) or a physiologically acceptable salt, formulation, or pro-drug thereof.

142. The method according to claim 138, wherein the LTB4 levels are measured in plasma after activation of leukocytes with calcium ionophore, and thereafter isolating the plasma.

143. The method according to claim 138, wherein the composition is administered with food.

144. The method according to claim 138, wherein the composition is administered on an empty stomach.

145. The method according to claim 138, wherein the human has been diagnosed as carrying a FLAP or LTA4H haplotype that correlates with increased risk of myocardial infarction.
